(19) United States
(12) Patent Application Publication
(10) Pub. No.: US 2026/0000327 A1
(43) Pub. Date: Jan. 1, 2026

(54) SYSTEM AND METHODS FOR DETECTING AND MITIGATING NEURO-DEFICIENCIES USING BRAIN IMAGING DATA

(71) Applicant: Matter Neuroscience Inc., Boulder, CO (US)

(72) Inventor: Axel BOUCHON, Kleinmachnow (DE)

(73) Assignee: Matter Neuroscience Inc., Boulder, CO (US)

(21) Appl. No.: 18/994,313

(22) PCT Filed: Jul. 28, 2023
(86) PCT No.: PCT/US2023/071174

§ 371 Date: Jan. 14, 2025

Related U.S. Application Data
(60) Provisional application No. 63/369,748, filed on Jul. 28, 2022.

(51) Int. Cl. A61B 5/16; A61B 5/00; A61B 5/377; G01R 33/48

(52) U.S. Cl. CPC A61B 5/165; A61B 5/0075; A61B 5/377; G01R 33/4806

(57) ABSTRACT

Disclosed herein are systems and methods for evaluating the underlying molecular (e.g., brain reward system molecules) cause for a depressive disorder and developing a treatment plan based on the identified molecular cause. In one or more examples, the systems and methods described herein can utilize any combination of brain imaging techniques, subjective emotion data taken from a patient, and/or one or more algorithms for translating emotional states to brain reward system molecules (e.g., neurotransmitters) to determine an underlying molecular cause for a depressive disorder of a patient. Once the underlying molecular cause of the depressive disorder of the patient is determined, a treatment plan that includes both behavioral and pharmacological aspects can be selected based on the identity of the reward system molecule that is determined to be deficient and the level of deficiency.

### CROSS-REFERENCE TO RELATED APPLICATIONS

This application claims the benefit of U.S. Provisional Application No. 63/369,748 filed Jul. 28, 2022, the entire contents of which are incorporated herein by reference.

### FIELD OF THE DISCLOSURE

This disclosure relates to systems and methods identifying the underlying molecular cause for a depressive disorder using brain imaging techniques, subjective emotion data, techniques for identifying distinct regions of interest indicative for distinct positive emotions, and techniques for translating recorded emotional states into brain reward system molecules.

### BACKGROUND OF THE DISCLOSURE

Mental health has become increasingly recognized as something that should be cared for with as much concern as physical health. Today, it is estimated that more than 80% of the worldwide population are burdened by depression, anxiety, or addiction at least once in their lives. This erosion of mental health has consequential implications for society at large. Mental health issues can be attributed to increases in crime, suicide rates, and even loss of productivity. Thus, improving mental health across a population not only confers benefits to the individual whose mental health is improved, but also benefits society.

Recognizing the importance of mental health, many approaches to improving mental health have been suggested and applied. For instance, philosophical, psychological, technical, and neuroscientific approaches to improving mental health have been implemented in isolation, focusing only on a single aspect of the disease (rather than holistically) with mixed success. Many of these approaches have failed or provided marginal improvements in mental health. The incidence and prevalence of depressive disorders have been skyrocketing in recent years with more than 260 million people impacted worldwide and a life-time incidence rate of >75%. Efficacy and effectiveness of available anti-depressants is historically very low with >70% of patients not experiencing any improvement or even progressing to a more severe disease state. The issue is further exacerbated by substantial deterioration of disease states after discontinuation of anti-depressants with significantly higher suicide rate observed in certain patient groups. Basic research has provided evidence that depression is caused by one or more dysfunctional reward systems. However, a precise method to identify the molecular basis for targeted intervention and leverage it for individualized therapy is missing. Thus, available pharmacological and behavioral therapies can't be properly matched to the underlying cause of disease leading to inefficient therapies. There is a substantial risk that the treatments approved and used today are actually making the disease worse over time since often times, conventional treatments are not properly addressing the underlying molecular impairment.

### SUMMARY OF THE DISCLOSURE

Accordingly, provided herein are systems and methods for evaluating the underlying molecular (e.g., brain reward system molecules) cause for a depressive disorder and developing a treatment plan based on the identified molecular cause. In one or more examples, the systems and methods described herein can utilize any combination of brain imaging techniques, subjective emotion data taken from a patient, and/or one or more algorithms for translating emotional states to brain reward system molecules (e.g., neurotransmitters) to determine an underlying molecular cause for a depressive disorder of a patient. In one or more examples, the subjective emotion data can be collected or received from a patient through an initial interview or other master data compilation regarding the patient's current and/or past mental condition. In one or more examples, subjective emotion data are collected systematically, for example using one or more of the techniques described in U.S. application Ser. No. 17/389,023, which is hereby incorporated by reference in its entirety. In one or more examples, the initial interview or master data compilation can include an assessment of the actual emotional wellbeing state by a subjective wellbeing self-assessment questionnaire. In one or more examples, the initial interview or master data compilation can include an assessment of the actual depression state of the patient by any clinical depression assessment questionnaire that allows for the categorization of mental states (e.g., HAMD17 or any other similar depression assessment questionnaire). In one or more examples, the patient's answers to the questionnaires or interview questions can be used to determine one or more neurotransmitter (e.g., molecular) deficiencies in the brain of the patient.

In one or more examples, the systems and methods described herein can also include applying one or more algorithms to convert a patient's recorded memories and emotion data (e.g., subjectively reported emotion data) in reactions to various events into neurotransmitter transmitter levels (e.g., reward system molecules), thus allowing for the identification of neurotransmitter deficiencies. In one or more examples, the algorithms can input a patient's emotional reactions to daily events and activities, translate the events/reactions into one or more neurotransmitter levels, and thus be used to identify one or more deficiencies in neurotransmitter levels.

In one or more examples, the system and methods described herein can include using brain imaging techniques such as functional magnetic resonance imaging (fMRI) and/or other similar techniques to determine deficiencies in neurotransmitter levels within a patient's brain. In one or more examples, a patient's brain can be imaged in real-time while the patient is being randomly exposed to photos and/or music that have emotional significance to the patient and thus are likely to cause reward center activity in the brain of the patient. In one or more examples, the signal intensity data from the brain scan and/or 3D location of brain activity can be used to identify distinct regions of interest in the brain that can be correlated with distinct emotions and/or reward system molecules (e.g., neurotransmitters).

In one or more examples, once the underlying molecular cause for the depressive disorder is identified (using any combination of the techniques described above), the systems and methods described herein can create a personalized anti-depression prevention and/or treatment protocol that is specifically tailored to the identified neurotransmitter deficiencies

In some embodiments, a first method for treating a neurological disorder is provided, the first method comprising: receiving information associated with a patient, wherein the received information includes information about one or more past events that the patient has engaged in and one or more emotions associated with the one or more events; determining one or more reward molecule levels of the patient based on the received information associated with the patient; receiving one or more scans of the patient's brain; determining one or more reward molecule levels of the patient based on the received one or more scans of the patient's brain; and determining one or more reward molecule deficiencies of the user based on the determined one or more reward molecule levels of the patient based on the received information associated with the patient and based on the determined one or more reward molecule levels of the patient based on the received one or more scans of the patient's brain.

In some embodiments of the first method, the method comprises selecting a predetermined treatment plan for the determined one or more reward molecule deficiencies based on an identity of each reward molecule deficiency,

In some embodiments of the first method, wherein the identity of the reward molecule deficiency is selected from a group consisting of: dopamine, serotonin, testosterone, oxytocin, cannabinoids, opioids.

In some embodiments of the first method, determining one or more reward molecule levels of the patient based on the received information associated with the patient comprises applying the received information associated with the patient to a positive emotion to a neurotransmitter (PE-NT) matrix.

In some embodiments of the first method, the received one or more scans of the patient's brain comprise real-time functional magnetic resonance imaging (rt-fMRI) scans.

In some embodiments of the first method, the received one or more scans of the patient's brain comprise electroencephalogram (EEG) scans.

In some embodiments of the first method, the received one or more brains scans are acquired using a method comprising; exposing the patient to one or more stimuli; determining one or more locations within the brain where a signal is generated in response to the stimuli; and determining a signal intensity associated with each location of the brain where a signal is generated in response to the stimuli.

In some embodiments of the first method, determining one or more reward molecule levels of the patient based on the received one or more scans of the patient's brain comprises: associating the one or more determined locations within the brain where a signal is generated in response to the stimuli to one or more types of reward molecules; and determining the one or more reward molecule levels of the patient based on the determine signal intensity associated each location of the brain where a signal is generated in response to the stimuli.

In some embodiments of the first method, the one or more emotions associated with the one or more events received information associated with the patient of the information associated with the patient comprises an emotion from the group comprising: enthusiasm, sexual desire, pride/recognition, nurturant love, contentment, attachment love, amusement, pleasure, and gratitude.

In some embodiments of the first method, the received information associated with a patient comprises the patient's responses to one or more questionnaires configured to diagnose depression in the patient.

In some embodiments, a second method for identifying and treating a neurological deficiency is provided, the method performed by a system comprising one or more processors, the second method comprising: receiving information associated with a patient, wherein the received information includes memory information about a plurality of patient memories, wherein, for each of the plurality of patient memories, the memory information indicates: a respective emotional intensity for each of one or more emotions, respective time information, and respective metadata indicating aspects of the memory; providing a plurality of stimuli to the patient, wherein each of the plurality of stimuli are respectively associated with one or more of the patient memories; while providing the stimulus to the patient, measuring brain activity of the patient, wherein measuring brain activity comprises collecting data indicating location of brain activity and associated intensity of brain activity; determining, based on the measured brain activity of the patient, a plurality of neurotransmitter levels for the patient; and identifying, based at least in part on the determined plurality of neurotransmitter levels for the patient, a neurological deficiency with respect to at least a first neurotransmitter for the patient.

In some embodiments, the second method comprises: identifying, based on the memory information and based on the identified neurological deficiency, a subset of the plurality of patient memories associated with a positive neurological response as indicated by the brain activity of the patient; identifying, based on the identified subset of the plurality of patient memories and based on the respective metadata indicating aspects for the identified subset, a first aspect that is common across a plurality of the memories; and generating and providing a personalized treatment plan for the patient comprising an indication of the first aspect.

In some embodiments of the second method, identifying the neurological deficiency comprises: based on the determined plurality of neurotransmitter levels for the patient, generating a provisional indication of the neurological deficiency; applying one or more rule sets based on pharmacological and/or clinical biochemical information regarding the patient to determine that the provisional indication of the neurological deficiency should not be negated.

In some embodiments of the second method, identifying the neurological deficiency comprises: based on the determined plurality of neurotransmitter levels for the patient, generating a provisional indication of the neurological deficiency; applying one or more rule sets based on the memory information for the patient, including by comparing emotional intensity information for the patient to emotional intensity threshold information, to determine that the provisional indication of the neurological deficiency should not be negated.

In some embodiments of the second method, identifying the neurological deficiency comprises: based on the determined plurality of neurotransmitter levels for the patient, generating a provisional indication of the neurological deficiency; and applying one or more rule sets based on the memory information for the patient, including by analyzing changes in emotional intensity information for the patient over time, to determine that the provisional indication of the neurological deficiency should not be negated.

In some embodiments of the second method, identifying the neurological deficiency comprises: based on the determined plurality of neurotransmitter levels for the patient, generating a provisional indication of the neurological deficiency; and applying one or more rule sets based on the memory information for the patient, including by applying the memory information to a positive emotion to a neurotransmitter (PE-NT) matrix, to determine that the provisional indication of the neurological deficiency should not be negated.

In some embodiments of the second method, the first neurotransmitter is selected from the group consisting of: dopamine, serotonin, testosterone, oxytocin, cannabinoids, and opioids.

In some embodiments of the second method, measuring brain activity of the patient comprises using a functional magnetic resonance imaging (fMRI) scan.

In some embodiments of the second method, the fMRI scan is a 7 Tesla fMRI scan.

In some embodiments of the second method, measuring brain activity of the patient comprises using an electroencephalogram (EEG) scan.

In some embodiments of the second method, measuring brain activity of the patient comprises using functional near-infrared spectroscopy (fNIRs).

In some embodiments of the second method, determining, based on the measured brain activity of the patient, the plurality of neurotransmitter levels for the patient comprises: determining one or more locations within the brain for which a signal is to be measured in response to the stimuli; determining one or more signal intensities, based on the measured brain activity of the patient, associated with each of the one or more locations of the brain; and determining the plurality of neurotransmitter levels based at least in part on the determined one or more locations and on the determined one or more signal intensities.

In some embodiments of the second method, determining the plurality of neurotransmitter levels comprises comparing a determined signal intensity for a determined location to a threshold signal intensity level for that location.

In some embodiments of the second method: the plurality of stimuli presented to the patient are analyzed based on the one or more emotions associated with the memories associated with the stimuli, as indicated in the received information, and determining the one or more locations within the brain is based at least in part on the one or more emotions indicated in the received information.

In some embodiments of the second method, the one or more emotions comprise an emotion from the group comprising: enthusiasm, sexual desire, pride/recognition, nurturant love, contentment, attachment love, amusement, pleasure, and gratitude.

In some embodiments, a system for identifying and treating a neurological deficiency is provided, the system comprising one or more processors configured to cause the system to: receive information associated with a patient, wherein the received information includes memory information about a plurality of patient memories, wherein, for each of the plurality of patient memories, the memory information indicates: a respective emotional intensity for each of one or more emotions, respective time information, and respective metadata indicating aspects of the memory; provide a plurality of stimuli to the patient, wherein each of the plurality of stimuli are respectively associated with one or more of the patient memories; while providing the stimulus to the patient, measure brain activity of the patient, wherein measuring brain activity comprises collecting data indicating location of brain activity and associated intensity of brain activity; determine, based on the measured brain activity of the patient, a plurality of neurotransmitter levels for the patient; and identify, based at least in part on the determined plurality of neurotransmitter levels for the patient, a neurological deficiency with respect to at least a first neurotransmitter for the patient.

In some embodiments, a non-transitory computer-readable storage medium storing instructions for identifying and treating a neurological deficiency is provided, the instructions configured to be executed by one or more processors of a system to cause the system to: receive information associated with a patient, wherein the received information includes memory information about a plurality of patient memories, wherein, for each of the plurality of patient memories, the memory information indicates: a respective emotional intensity for each of one or more emotions, respective time information, and respective metadata indicating aspects of the memory; provide a plurality of stimuli to the patient, wherein each of the plurality of stimuli are respectively associated with one or more of the patient memories; while providing the stimulus to the patient, measure brain activity of the patient, wherein measuring brain activity comprises collecting data indicating location of brain activity and associated intensity of brain activity; determine, based on the measured brain activity of the patient, a plurality of neurotransmitter levels for the patient; and identify, based at least in part on the determined plurality of neurotransmitter levels for the patient, a neurological deficiency with respect to at least a first neurotransmitter for the patient.

Any one or more of the features of the above-listed embodiments may be combined, in whole or in part, with one another and/or with any other disclosure herein.

### BRIEF DESCRIPTION OF THE FIGURES

FIG. 1 illustrates an exemplary process for determining molecular deficiencies in the brain of a patient according to examples of the disclosure.

FIG. 2 illustrates an exemplary neurotransmitter level calculation according to examples of the disclosure.

FIG. 3 illustrates an exemplary process for determining neurotransmitter levels in the brain of a patient according to examples of the disclosure.

FIG. 4 illustrates an exemplary process for determining a treatment plan based on a determined molecular deficiency according to examples of the disclosure.

FIGS. 5A-5B illustrates an exemplary method for identifying and treating a neurological deficiency.

FIGS. 6A-6F illustrate exemplary brain scans indicating regions of interest and correlated emotions, reward systems, and/or neurotransmitters.

FIGS. 7A-C illustrates exemplary calculations performed on memory information.

FIG. 8 illustrates an example of a computing device in accordance with one embodiment.

### DETAILED DESCRIPTION OF THE DISCLOSURE

In the following description of the disclosure and embodiments, reference is made to the accompanying drawings in which are shown, by way of illustration, specific embodiments that can be practiced. It is to be understood that other embodiments and examples can be practiced, and changes can be made, without departing from the scope of the disclosure.

In addition, it is also to be understood that the singular forms “a,” “an,” and “the” used in the following description are intended to include the plural forms as well unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Some portions of the detailed description that follow are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps (instructions) leading to a desired result. The steps are those requiring physical manipulations of physical quantities. Usually, though not necessarily, these quantities take the form of electrical, magnetic, or optical signals capable of being stored, transferred, combined, compared, and otherwise manipulated. It is convenient at times, principally for reasons of common usage, to refer to these signals as bits, values, elements, symbols, characters, terms, numbers, or the like. Furthermore, it is also convenient at times to refer to certain arrangements of steps requiring physical manipulations of physical quantities as modules or code devices without loss of generality.

However, all of these and similar terms are to be associated with the appropriate physical quantities and are merely convenient labels applied to these quantities. Unless specifically stated otherwise as apparent from the following discussion, it is appreciated that, throughout the description, discussions utilizing terms such as “processing,” “computing,” “calculating,” “determining,” “displaying,” or the like refer to the action and processes of a computer system, or similar electronic computing device, that manipulates and transforms data represented as physical (electronic) quantities within the computer system memories or registers or other such information storage, transmission, or display devices.

Certain aspects of the present Disclosure include process steps and instructions described herein in the form of an algorithm. It should be noted that the process steps and instructions of the present Disclosure could be embodied in software, firmware, or hardware, and, when embodied in software, they could be downloaded to reside on and be operated from different platforms used by a variety of operating systems.

The present disclosure also relates to a device for performing the operations herein. This device may be specially constructed for the required purposes, or it may comprise a general-purpose computer selectively activated or reconfigured by a computer program stored in the computer. Such a computer program may be stored in a non-transitory, computer-readable storage medium such as, but not limited to, any type of disk, including floppy disks, optical disks, CD-ROMs, magnetic-optical disks, read-only memories (ROMs), random access memories (RAMs), EPROMS, EEPROMs, magnetic or optical cards, application-specific integrated circuits (ASICs), or any type of media suitable for storing electronic instructions and each coupled to a computer system bus. Furthermore, the computers referred to in the specification may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present disclosure is not described with reference to any particular programming language. It will be appreciated that a variety of programming languages may be used to implement the teachings of the present disclosure as described herein.

Depression or other neurological disorders can often be attributed to dysfunctional reward systems in the brain of a patient, wherein the brain is often failing to produce adequate brain reward system molecules associated with stable moods. Often times, anti-depressant medication is prescribed to a patient to treat a mood disorder, however such treatments often don't provide satisfactory outcomes, with patients often not experiencing any improvement in their condition, and in some cases exacerbating their condition when they eventually discontinue the medication. While it is known that lack of certain brain reward system molecules can be at the root cause of certain mood disorders, precisely identifying which neurotransmitter/molecule is deficient, and then using such knowledge to generate a treatment plan for the patient is lacking. This deficiency means that behavior and pharmacological therapies to treat mood disorders can't be matched to the underlying cause of the mood disorder, thus leading to poorer outcomes for patients with the mood disorder.

As described above, the first step in generating effective therapies (behavioral and pharmacological) for the treatment of mood disorders can be to accurately identify any deficiencies in the reward molecules that may serve as the underlying cause of the mood disorder. For instance, mood disorders (e.g., depression) can often be linked to deficiencies in the brain of various reward molecules including but not limited to dopamine, serotonin, testosterone, oxytocin, cannabinoids, and opioids. Detecting deficiencies in these reward molecules can be challenging. Thus, and as described in detail below, a method for detecting deficiencies in reward molecules is presented that can combine various diagnostic tools to accurately detect deficiencies in reward molecules.

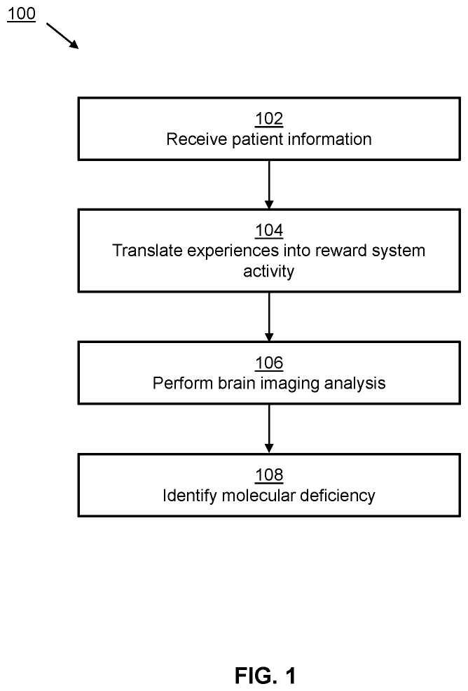

FIG. 1 illustrates an exemplary process for determining molecular deficiencies in the brain of a patient according to examples of the disclosure. In one or more examples, the process 100 of FIG. 1 can begin at step 102 wherein information regarding the patient's mental is received. In one or more examples, the received information can include a patient's response to one or more entry interviews wherein the patient is asked a series of questions (either verbally or in written form) regarding their emotional well-being state, their actual depression state through the use of clinical depression assessment questionnaires such as the Hamilton Depression Ration Scale (HAMD17) or any commensurate diagnostic questionnaire, as well as their substance dependencies/addictions/abuse or behavior dependencies/addictions/abuse.

In one or more examples, the entry interview/questionnaire responses received at step 102 can provide a first indication for potential reward system deficits and/or unsatisfied needs. In one or more examples, at step 102, the patient/participant can also be guided to assess and record her/his life's peak-positive-memories by rating the entire spectrum of positive emotions and selecting a representative photo or music triggering a memory recall. In one or more examples, this spectrum can include, but is not limited to, one or more of the following emotions: enthusiasm, sexual desire, pride/recognition, nurturant love (alternatively referred to as family love), contentment, attachment love (alternatively referred to as friendship love), amusement, pleasure, and gratitude. In one or more examples, and for each emotion, the patient/participant can be asked to record a pre-defined number of peak memories from their life that is correlated to each emotion. In one or more examples, in addition to past memories correlating to the emotions, information about the patient's current memories can also be received. For instance, in one or more examples, the patient can be asked to assess and record real-time memories for a pre-defined period of time (e.g., 30 days) and asked to record memories that elicit emotions associated with emotional well-being. In one or more examples, the user can be asked to characterize the emotion felt (e.g., enthusiasm, sexual desire, pride/recognition, nurturant love, contentment, amusement attachment love, pleasure, and gratitude) as well as the level of intensity that each emotion was felt.

In one or more examples, the information received at step 102 can be used to determine reward system molecule deficiencies. For instance, using the information about the patient received at step 102, the process 100 at step 104 can translate the user's experiences and emotional responses to the one or more experiences into reward molecules and reward system activity using one or more algorithms that are specifically configured to translate emotions tied to experiences into reward system molecule activity with the purpose of identifying any gaps in reward system molecules.

One exemplary algorithm that can be employed to translate the emotions recorded by the user (alternatively referred to herein as the “patient”) into neurotransmitter levels (e.g., reward system molecules) can include the use of a positive emotion to Neurotransmitter (PE-NT) matrix that can translate the quantified positive emotions derived at step 102 into an amount of each of the neurotransmitters associated with positive emotions. In one or more examples, and for the purposes of the present disclosure, the term “neurotransmitter” can refer to specific neurotransmitters that are associated the brain's reward system. In one or more examples, and for the purposes of the present disclosure, the calculation can be based on the user information received at step 102.

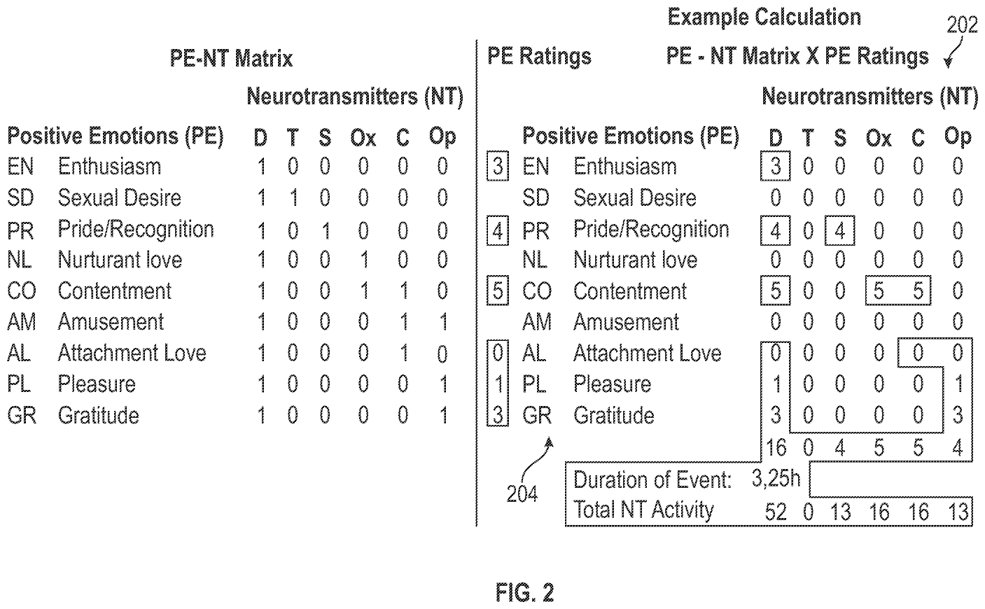

FIG. 2 illustrates an exemplary neurotransmitter level calculation according to examples of the disclosure. The example of FIG. 2 illustrates an exemplary PE-NT matrix 202 that can be used to calculate the amount of a particular neurotransmitter based on a patient's recorded emotions and intensity of emotion correlated with a particular memory or event. In one or more examples, the rows of the PE-NT matrix 102 can represent the emotion categories such as enthusiasm, sexual desire, pride/recognition, nurturant love, contentment, amusement, pleasure, and gratitude. In one or more examples, the columns of the PE-NT matrix 202 can represent the brain reward system molecules (dopamine, testosterone, serotonin, oxytocin, cannabinoids, and opioids) associated with positive emotions. A “1” in the matrix can indicate that a particular neurotransmitter is associated with that particular emotion. A “0” in the matrix can indicate that a particular neurotransmitter is not associated with that particular emotion.

In one or more examples, the PE-NT matrix 202 can be used to calculate the amount of a neurotransmitter associated with an activity. Calculation 204 illustrates an exemplary calculation. In one or more examples, the calculation 204 can include a PE ratings column that shows the ratings provided by the user at step 102 of FIG. 1. For instance, when rating their emotions pertaining to a particular memory or event, the user may have rated their enthusiasm during the event as mild (indicating that it is lower than average but still present) and so that rating can be quantified as a 3. In the same example, in the PE ratings column the user may rate their contentment as a 5 which is average. In order to calculate the amount of neurotransmitter, the calculation can multiply the rating by the PE-NT matrix to generate a number associated with the amount of a particular neurotransmitter. For instance, as shown in PE-NT matrix 202, enthusiasm can be associated with the release of dopamine. Looking at calculation 204, the PE rating for enthusiasm (provided by the user) is 3. That value is multiplied by 1 under the dopamine column to arrive at a value of 3. Thus, with respect to the enthusiasm felt by the user as expressed in the PE rating, the dopamine level associated with that enthusiasm is quantified at 3. In one or more examples, the remaining columns are left at 0 because those brain reward system molecules are not associated with enthusiasm.

In one or more examples, each and every positive emotion can be multiplied by the PE rating to arrive at a value for each neurotransmitter. For instance, contentment was rated 5 by the user, and thus since contentment is associated with dopamine, oxytocin, and cannabinoids, each of those elements can be set at 5 (multiplying 5×1). Once the calculation is made for each emotion at each neurotransmitter, the calculation can add up the totals for each neurotransmitter. Turning to the example calculation 204 of FIG. 2, for dopamine, the dopamine levels for each emotion can add up to 16. For serotonin, the calculation can add up to 4 and so on and so forth for each neurotransmitter.

Once each neurotransmitter is tallied up, each tally can be multiplied by the duration of the event (assuming the user has provided that information) to determine the total neurotransmitter activity associated with the event. Using the example of dopamine, the tally added up to 16 (as described above) and that tally can be multiplied by 3.25 (e.g., 3.25 hours which was the time indicated by the user that the event lasted) to come to a total of 52 for dopamine. Each individual level for each neurotransmitter can be similarly calculated for the given memory. (In some embodiments, multiplication by time may not be used. In some embodiments, neurotransmitter levels may be normalized to one hour, e.g., multiplied by one.)

The neurotransmitter level determinations for each memory may be aggregated. The aggregated neurotransmitter level data may be used to calculate neurotransmitter deficiencies. For example, neurotransmitter deficiencies may be identified by using the aggregated neurotransmitter level data to compare neurotransmitter levels over time.

In one or more examples, the exemplary algorithm described above with respect to FIG. 2 for translating emotional responses to memories and events into reward molecule/neurotransmitter levels is meant for purposes of example only and should not be construed as limiting to the disclosure. In one or more examples, any algorithm that can translate emotional experiences into neurotransmitter levels can be utilized at step 102 of process 100.

In one or more examples, and returning to the example of FIG. 1, in order to gather further data regarding reward molecule levels in the brain, the process 100 can include step 106 wherein brain imaging is used to determine whether there are any reward molecule deficiencies associated with the patient. In one or more examples, and as described in further detail below, a patient can be presented with various stimuli (e.g., music and photos) that are known to elicit positive emotions from the user. While the patient is being stimulated, in one or more examples, the patient's brain can be scanned to record their brain activity. In one or more examples, the brain activity observed in the scans can be used to determine reward molecule deficiencies. In one or more examples, the exemplary calculation described above with respect to FIG. 2 can be refined with time using feedback information from brain imaging scan data (described in further detail below.) For instance, information gleaned from brain scans can be used to refine the multipliers used in the calculation.

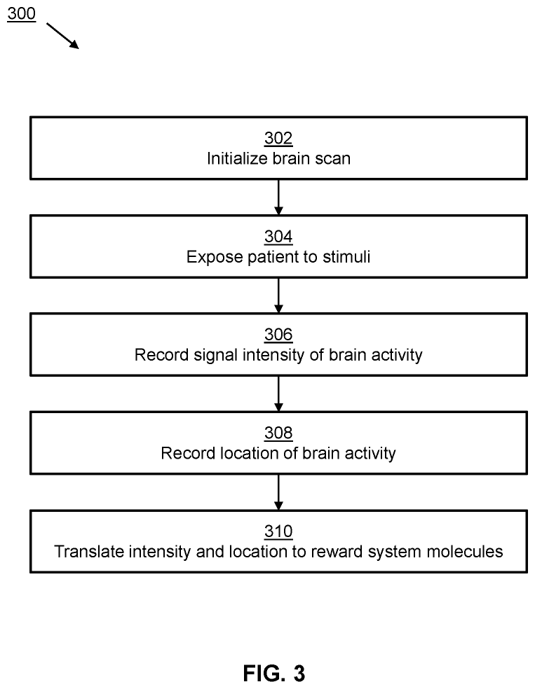

FIG. 3 illustrates an exemplary process for using brain imaging to determine molecular deficiencies in a patient's brain according to examples of the disclosure. In one or more examples, the process 300 of FIG. 3 can be utilized to identify reward molecule deficiencies from brain imaging data. In one or more examples, and as described in further detail below, the process 300 can be used in conjunction with, alternatively to, and/or in parallel with determinations of reward molecule deficiencies based on self-reported patient data and/or other patient data aside from brain scan data. In one or more examples, the process 300 of FIG. can begin at step 302 wherein a brain scan of the patient is initialized. In one or more examples, a “brain scan” can refer to a plurality of brain diagnostic tools that are configured to record brain activity in real-time or near-real time. For instance, in one or more examples, a brain scan can include functional magnetic resonance imaging (“fMRI”), real-time functional magnetic resonance imaging (“rt-fMRI”), real-time functional magnetic resonance imaging neurofeedback (rt-fMRI-NF), a full-scalp electroencephalogram (“EEG”), and/or full-scalp functional near-infrared spectroscopy (fNIRS). The above brain imaging procedures/tools are meant as examples only and should not be construed as limiting to the disclosure. Any tool/method configured to measure brain activity in response to stimuli can be considered a “brain scan” within the scope of the present disclosure.

In one or more examples, once the brain scan has been initialized at step 302, the process 300 can move to step 304 wherein the patient is exposed to stimuli that has be a priori identified by the patient as having previously elicited positive emotions associated with the release of reward activity molecules in the brain. In one or more examples, the stimuli can include auditory language stimuli such as spoken or recorded messages, written language stimuli such as displayed text, music (e.g., audio stimulation), and/or photos/videos (e.g., pictures of persons or places). The stimuli may relate to positive memories of the patient either directly or indirectly (e.g., by containing a common content (e.g., a common person or place) as a patient memory). In one or more examples, the stimuli applied at step 304 can be extracted from the patient data received at step 102 in process 100 of FIG. 1.

In one or more examples, the stimuli applied to the patient at step 304 can be designed to elicit the release of reward activity molecules in the brain. This in turn triggers brain activity that can be detected and measured by the brain scan in distinct regions of interest within the brain. Thus, in one or more examples, while the stimuli are being applied to the patient, the signal intensity of the brain activity elicited by the stimuli can be measured at step 306 using the brain scanning tool. In one or more examples, in addition to measuring signal intensity, at step 308, the three-dimensional location of the brain activity elicited by the stimuli can also be recorded. In one or more examples, the signal intensity and three-dimensional location of the brain activity (as extracted from the brain scan) can be used to identify activity in pre-defined reward centers in the brain that can be correlated with emotions and reward system molecules. Thus, in one or more examples, the signal intensity and the location of the signal recorded at step 306 and 308 respectively, can be translated into reward system molecules levels. In this way, gaps in certain reward molecules determined by the algorithms described above with respect to FIG. 2 can be further confirmed by brain imaging data using the process 300 of FIG. 3.

In one or more examples, the areas of the brain that correlate or are associated with various reward molecules can defined by employing a brain study that is configured to map emotions and reward system activity while the brain is experiencing memory recall so as to create a predictive map of the entire reward system landscape. A reward map can be created through experimentation. In one or more examples, a brain study can be performed on each individual patient to create a predictive map of the individual patient's reward system landscape. In one or more examples, a brain study can be conducted on multiple control subjects (for example 1-5). In one or more examples, the brain study is performed on subjects who have a fully prepared memory and emotion database (memories associated with positive emotions) that can include a plurality of person positive memories and can have one or more peak memories (e.g., memories that elicited the greatest emotional response) are selected to create a reward activity map. In one or more examples, each memory (e.g., audio/visual stimuli such as photos and/or music) can be pre-rated by the patient for total emotional intensity as well as the intensity of the positive emotions described above (e.g., enthusiasm, sexual desire, pride/recognition, nurturant love, contentment, amusement, attachment love, pleasure, and gratitude). For instance, in one or more examples, the memories can be rated on scale of 0-8, with 0 meaning the emotion was not experienced at all, with 8 representing the highest experience of that emotion ever in the subject's life. In one example, before or while memory recall is performed, patients and/or control subjects can be treated with chemical or biological agonists/antagonists specific to one or more reward system molecule pathways, thus allowing the selective enhancement or inhibition of each of the reward system pathways specifically, allowing for causal links for neurotransmitters to be better determined.

In one or more examples, the subject(s) will be shown a randomized collection of photos/stimuli with some of the stimuli coming from the pre-defined memories pool, while others are control stimuli (for instance in the form of white noise or blank pictures). In one or more or examples, as the subject is shown the randomized photos, both an EEG and an fMRI brain scan of the subject is recorded to determine if a signal is detected in either scan in response to the stimuli. In one or more examples, any differences between the signal intensities of the EEG and fMRI in correlation with the pre-rated total emotion intensity can be detected. For example, differences between signal intensity levels for fMRI data as compared to expected fMRI signal intensity data based on subjective emotional intensity reporting may be analyzed; differences between signal intensity levels for EEG data as compared to expected EEG signal intensity data based on subjective emotional intensity reporting may be analyzed; and/or differences between fMRI signal intensity data and EEG signal intensity data (e.g., normalized based on typical respective signal intensities) may be analyzed. Additionally, the scans can be analyzed to determine distinct signal patterns in both the EEG and the fMRI correlating with the pre-identified emotions. In one or more examples, patterns can be identified in the EEG data by examining the fast Fourier transform (FFT) of epoched EEG signals. Additionally or alternatively, the patterns can be identified by perform frequency-band power analysis (theta, alpha, beta, and gamma) across brain regions: frontal, central, parieto-occipital, left temporal, right temporal, frontal midline, etc. Additionally or alternatively, power ratio analyses across frontal electrodes of the EEG (theta/beta ratio, alpha/beta ratio) can be used to detect patterns in the EEG that can help to identify reward centers in the brain. Additionally or alternatively, power asymmetries across pre-frontal electrodes: theta, alpha, bet, gamma can be utilized to detect patterns in the EEG data. In one or more examples, and additionally or alternatively, visual comparisons can be employed that compare intensity of signals across all emotions vs. control stimuli. Additionally or alternatively, a comparison of stimuli of each emotion can be used to detect patterns, as well as a comparison of control stimuli vs. positive emotional stimuli (across all emotions and intensities).

In one or more examples, and similar to the EEG, the fMRI data associated with the patient receiving the randomized stimuli can also be used to detect the signal intensities and locations in the brain that produce activity in response to the stimuli provided to the patient during the study. In one or more examples, the fMRI detection and analysis can be utilized in accordance with the techniques discuss in line with techniques described in “Value Signals in the Prefrontal Cortex Predict Individual Preferences across Reward Categories” by Gross et al. (The Journal of Neuroscience, May 28, 2014⋅34(22):7580-7586.) In one or more examples, the fMRI may be performed at 3 Tesla (3T). In one or more examples, the fMRI may be performed at 7 Tesla (7T) or higher. In one or more examples, the fMRI may be performed at 14 Tesla (14T).

In one or more examples, the process 300 can be utilized by itself or in conjunction with subjective data regarding emotional associations with the various memories, such as the self-reported data provided by the patient. The process 300 can be used in either manner to detect one or more gaps or deficiencies in the patient's brain that may be causing a depressive disorder. Returning to the example of FIG. 1, in one or more examples, once the brain imaging analysis is completed at step 106, the process 100 can move to step 108 wherein one or more molecular deficiencies are identified based on the results of step 104 and/or step 106 of process 100. In one or more examples, the gaps or deficiencies identified at step 106 based on the data gathered from steps 102 and/or 104 can be used to prepare a personalized plan for treatment that can be responsive to the root cause of the patient's depression or mood disorder. Thus, in one or more examples, the gap analysis obtained from subjective assessment and confirmed by brain imaging can be used to design a personal depression prevention/treatment protocol. In one or more examples, the protocol can cover the entire reward system landscape beyond the existing repertoire of approved anti-depressants and known behavioral therapies that are in use today. In one or more examples, to reduce the risk of pharmacological dependencies and disease relapse, each therapy created for a determined deficiency in reward molecules can use behavioral and pharmacological components addressing all reward system gaps identified. The former guides the patient/participant towards behaviors intrinsically filling a distinct, personal reward system gap-such behavioral corrective actions are the anchor for a long-lasting therapeutic effectiveness. The latter can extrinsically support the patient/participant with the lacking, active reward system molecule thus, accelerating improvement.

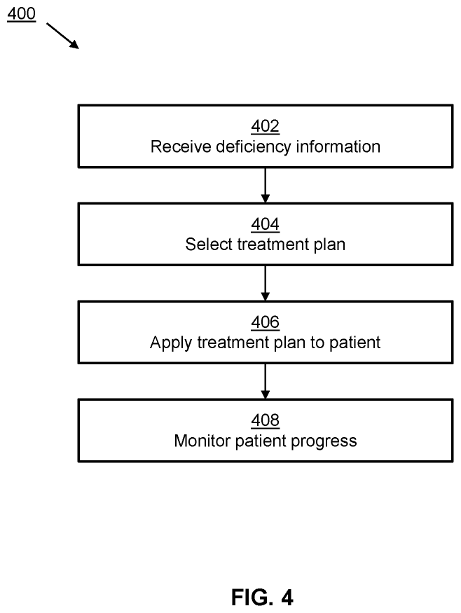

FIG. 4 illustrates an exemplary process for determining a treatment plan based on a determined molecular deficiency according to examples of the disclosure. In one or more examples, the process 400 of FIG. 4 can begin at step 402, wherein one or more reward molecule deficiencies pertaining to a patient (ascertained for instance using the process 100 of FIG. 1) can be received. As described above, in one or more examples reward molecules can include, but are not limited to, dopamine, serotonin, testosterone, oxytocin, cannabinoids, or opioids. Thus, in one or more examples, step 402 can include receiving information that identifies one or more reward molecule deficiencies but can also include receiving a quantification of the amount of the deficiency so that the severity of the deficiency can also be assessed.

In one or more examples, once the information pertaining to the deficiency has been received at step 402, then in one or more examples the process 400 of FIG. 4 can move to step 404 wherein a treatment plan based on the identified deficiency can be selected. In one or more examples, and as described above, a treatment plan can include both a pharmacological treatment plan as well as a behavioral treatment plan that individual and/or collectively can be configured to remedy the reward molecule deficiency identified by step 402. In one or more examples, since each reward molecule manifests its own pharmacological and behavioral response. In one more examples, the treatment plan can be specifically tailored to the identified deficiency. A specifically tailored treatment plan that can be personalized to each individual patient provides a more effective method of treatment than currently administered generalized treatment plans.

As an example, an exemplary treatment plan for dopamine deficiency is presented below. As a central cofactor for all positive emotions, a hallmark of a Dopamine deficiency can a full set of peak-memories covering all emotions equally. The selected peak memories may be from several years in the past; selected peak memories may cover the entire life of the subject, from recent memories (e.g., a few days or weeks prior) to memories from several years in the past. In contrast, actual memories may show an overall low rating on emotional significance and a below-average rating across all emotion intensities. Single highly rated memories show a high probability of activities with a high adrenaline stimulating factor given its synergistic effect to Dopamine, e.g., bungee jumping, parachuting, and excessive partying. Among individuals with a dopamine gap, acute or historical substance use or abuse is primarily focused on stimulants, such as Amphetamine (ADDERALL), Methylphenidate (RITALIN), Cocaine or methamphetamine. Individuals suffering from dopamine deficiency often report substantial deep depressive periods after using/abusing stimulant drugs. High amounts of caffeine intake from nutrition can be another indicator of dopamine deficiency. Acute or historical behavioral abuse is primarily focused around online gaming or gambling with very short gap between small digital rewards triggering dopamine release.

Structural MRI (sMRI) shows a lower rGMV in the rIC and rPC compared to controls. In response to past peak-memories, functional MRI (fMRI) shows activity in all brain regions carrying value-signals, such as right-anterior medial prefrontal cortex (mPFC), right-dorsal mPFC, right mPFC, left-dorsal PFC, left mPFC, left-anterior PFC and anterior cingulate cortex. In contrast, in response to actual memories, signal intensity is significantly reduced across all these areas. The number of peak memories that were recorded under the support of stimulating drugs can provide an indication on the respective dopamine gap (e.g., by comparing fMRI intensity of drug-supported peak memories versus non-drug-supported peak memories).

Thus, in one or more examples, given the issues associated with dopamine deficiency described above, activities that contain a mental and/or physical learning component with small but quick improvement and reward cycles should be applied for behavioral therapy, (e.g., boulder climbing, any crafted work, new language learning in a local environment). If needed, pharmacological therapy should be focused on medical Amphetamine or Methylphenidate. Classical anti-depressants of all classes, THC and substances stimulating the CB1/2 systems are contraindicated in this patient group. In one or more examples, testosterone therapy may not be supportive or effective unless testosterone levels are below base line. Co-treatment of amphetamine or methylphenidate with anti-depressants should only be used if there is a gap in the serotonin reward system, too. In case of recreational drug addiction to cocaine, methamphetamine, or similar it is critical to first complete a rehabilitation to complete withdrawal.

In one or more examples, an exemplary treatment plan for testosterone deficiency is provided below. An activity gap of the testosterone reward system can have two causes: (i) the threshold to trigger a testosterone stimulation is normal but available testosterone levels are too low due to impairment of natural testosterone production (mainly driven by stress or aging) or (ii) the threshold to trigger a testosterone stimulation is abnormally high while testosterone levels are normal (mainly driven by high pornography abuse, compulsive sexual behavior, hypersexuality disorder sex or sex addiction). To distinguish the two causes and patient groups, the memory analysis described above can provide the critical insights. In case general sexual desire and activity is low/zero (below-average number of peak memories and actual memories rated for sexual desire) in the patient/participant and pornography use is low/zero, a testosterone blood test should be initiated. In one or more examples, if testosterone blood levels are below base levels, supplementation with testosterone gels or injections can be initiated instantly and can correct the depressive disorder quickly. Classical anti-depressants of all classes, THC and substances stimulating the CB1/2 systems or any stimulants are contraindicated in patients whose threshold to trigger a testosterone stimulation is normal but available testosterone levels are too low due to impairment of natural testosterone production. It should be of note that in this patient group, brain imaging results using fMRI, sMRI, EEG or fNIRS can hardly be distinguished from normal individuals and can therefore not be used for differential diagnosis.

In contrast, patients for whom the threshold to trigger a testosterone stimulation is abnormally high while testosterone levels are normal showing a disturbed Testosterone threshold, but normal Testosterone levels show an above-average number of peak memories covering sexual activities with very high emotions ratings across all emotions for such activities. Dependent on the level of depression, actual memories are absent of any memories covering sexual activities or very few memories rated very high on the experience of sexual desire. Brain imaging using fMRI, sMRI, EEG or fNIRS provides a similar pattern to patients with a Dopamine gap therefore providing limited input for a differential diagnosis. In one or more examples, these patients can benefit from classical anti-depressants such as SSRIs with a very high probability. Stimulants of any kind may be contraindicated. In one or more examples, behavioral therapy should focus the patient initially towards physical activities, primarily individual endurance sports such as running, cycling, hiking, and climbing; intense weight training should be avoided. In severe cases, classical addiction therapy can be applied in parallel.

In one or more examples, an exemplary treatment plan for serotonin deficiency is provided below. A hallmark of a serotonin deficiency can be a full set of peak-memories covering all emotions equally. However, memories with high ratings in pride/recognition are recorded primarily during childhood and are tied to positive parental feedback most of the times. Actual memories display a very low number of memories with a pride/recognition experience and the ones showing high ratings for pride/recognition are skewed towards experiencing pride/recognition for others (kids, friends) instead of self-pride/recognition.

In one or more examples, among individuals with a serotonin gap, acute or historical recreational substance use or abuse is focused on 3,4-Methylenedioxymethamphetamine (ecstasy, MDMA), Ketamine and/or psychedelics. In addition, these individuals can report significant withdrawal symptoms after using MDMA and ecstasy. In one or more examples acute or historical behavioral dependency can be primarily focused around social media. In addition, there is a higher incidence in this group for sport addictions and eating disorders. In one or more examples, Structural MRI (sMRI) can show a significantly lower rGMV in the rIC and rPC compared to controls. In one or more examples, and in response to past peak-memories, functional MRI (fMRI) can show activity in all brain regions carrying value-signals, such as right-anterior medial prefrontal cortex (mPFC), right-dorsal mPFC, right mPFC, left-dorsal PFC, left mPFC, left-anterior PFC and anterior cingulate cortex. In one or more examples, a particularly strong activity response across all brain regions can be detected with selected, highly rated memories using self-portraits (“Selfie”) as recall trigger for self-pride/recognition. In one or more examples, this test can be a critical differentiator for diagnosis. The number of peak memories that were recorded under the support of above-mentioned recreational drugs can provide an indication on the respective serotonin gap (e.g., by comparing fMRI intensity of drug-supported peak memories versus non-drug-supported peak memories).

Based on the identified deficiencies in serotonin activities that focus on play, playful learning, non-competitive group actions that are task oriented would be indicated for behavioral therapy, e.g., group activities in and for nature or helping other people, travel, nature preservation. Activities with strong competitive focus are contraindicated unless they are team challenges against a physical objective, e.g., white water rafting, rowing, climbing/rope team, and scuba diving. Theater play or playing in an orchestra is also indicated. If needed, pharmacological therapy should focus on anti-depressants targeting the Serotonin reward system only, such as SSRIs. These substances have a high efficacy in this target audience. Medical Ketamine, THC and substances stimulating the CB1/2 systems should are highly effective, too. In contrast, antidepressants also targeting the dopamine pathway as well as any stimulants may be strictly contraindicated. Co-treatment of Amphetamine or Methylphenidate with anti-depressants should only be used in rare cases if there is a gap in the Dopamine reward system, too. In one or more examples, in case of a recreational drug addiction to any substance it is critical to first complete a rehab to complete withdrawal. In one or more examples, during therapy, monitoring would focus on the increase of fMRI intensity for actual memories focusing on experiencing self-pride/recognition and increasing the number of peak memories created without stimulating drug use. Over time, pharmacological therapy should be phased-out in line with progress of behavioral therapy. In one or more examples, monitoring can be used to provide continuous guidance during phase-out of pharmacological therapy. Phase-out is reverted, slowed, or stopped in case fMRI indicators stagnate or worsen.

In one or more examples, an exemplary treatment plan for oxytocin deficiency is provided below. A hallmark of an oxytocin deficiency can be a full set of peak-memories covering all emotions equally. However, memories with high ratings in nurturant love as well as contentment are recorded primarily during childhood and with above-average frequency include pets. Actual memories (e.g., more recent memories) display a very low number of memories with a nurturant love or contentment experience. The ones showing high ratings for nurturant love are very often tied to pets or children. Among individuals with an oxytocin gap, acute or historical recreational substance use or abuse is very rare, if at all focused on few experiences with 3,4-Methylenedioxymethamphetamine (ecstasy, MDMA). For the differentiation to patients with a Cannabinoid reward system gap it can be important to note that patients with an oxytocin impairment are rarely using/abusing THC-containing recreational drugs, such as cannabis or marijuana. In one or more examples, acute or historical behavioral dependencies are rare, too. However, low levels of oxytocin can be associated with a higher incidence rate for autisms and anorexia nervosa what is identified during the initial interview. Structural MRI (sMRI) shows a lower rGMV in the rIC and rPC compared to controls. In response to past peak-memories, functional MRI (fMRI) shows activity in all brain regions carrying value-signals, such as right-anterior medial prefrontal cortex (mPFC), right-dorsal mPFC, right mPFC, left-dorsal PFC, left mPFC, left-anterior PFC and anterior cingulate cortex. A particularly strong activity response across key brain regions associated with trust and social interaction can be detected upon recall of memories rating high for nurturant love. This test is a critical differentiator for diagnosis.

In one or more examples, a treatment plan for an identified oxytocin deficiency can be focused on behavioral therapy given the limitation of pharmacological therapies available. (However, a pharmacological therapy including oxytocin spray could be used, e.g., to improve depression symptoms.) In the behavioral therapy, activities should focus on nurturant love-supporting actions as they are highly effective, e.g., pets/puppy playing, joint meals, involve family members in therapy (if perceived positive by the patient), team sports with benign physical contact (dancing, basketball, horseback riding, ice skating (team), sailing, tandem parachuting). Pharmacological therapy for oxytocin deficiency may be limited to Oxytocin nasal sprays that show an immediate positive effect on depressive symptoms in this patient group. In one or more examples, all other anti-depressants and stimulants may be strictly contraindicated as they could worsen the condition. In one or more examples, monitoring can be focused on increased brain activity in brain regions associated with trust and social interaction detected by fMRI and increasing the number of actual memories with an above-average rating in nurturant love and contentment.

In one or more examples, a treatment plan for cannabinoid deficiency is provided below. A hallmark of a cannabinoid deficiency can be a full set of peak-memories covering all emotions. However, memories with high ratings in attachment love and in particular amusement are recorded primarily during childhood or in a distinct past. In one or more examples, the absence of below-average ratings for the experience of amusement can be a critical indicator for a Cannabinoid-impairment. In one or more examples, actual memories may display a very low number of memories with an attachment love or amusement experience. The number of memories that were recorded under the support of THC-containing drugs can provide an indication on the respective Cannabinoid gap (compare fMRI intensity of drug-supported peak memories versus non-drug-supported peak memories).

Among individuals with a cannabinoid gap, acute or historical recreational substance use or abuse of THC-containing product, such as cannabis or marijuana can be very high. In one or more examples, this can be a critical differentiation factor for the differentiation to patients with an oxytocin reward system gap. Acute or historical behavioral dependencies are focused on digital social media and games with deep and long connections, e.g., multiplayer strategic games, and expert forum activities. In one or more examples, structural MRI (sMRI) shows low rGMV in the rIC and rPC compared to all other patient groups and controls. This can be a critical factor for differential diagnosis. In one or more examples, in response to past peak-memories, functional MRI (fMRI) can show activity in all brain regions carrying value-signals, such as right-anterior medial prefrontal cortex (mPFC), right-dorsal mPFC, right mPFC, left-dorsal PFC, left mPFC, left-anterior PFC and anterior cingulate cortex. Similarly to patients with an Oxytocin gap, a particularly strong activity response across key brain regions associated with trust and social interaction can be detected upon recall of memories rating high for attachment love and amusement. In one or more examples, the above test can be a critical differentiator for diagnosis versus Dopamine-, Testosterone-, Serotonin- and Opioid-impairments.

In one or more examples, treatments for a determined cannabinoid deficiency can include activities that focus on group actions supporting laughter and the experience of friendship (e.g., attachment) and belonging. In one or more examples, play can be more important than competition and winning but a competitive component can indicate if it is focused on team competitions, e.g., all (non-professional) team sports, all creative activities in a group/team, play, playing music together/orchestra, active group vacation & tasks, small business activities with friends. Digital social media activity is reduced stepwise and substituted with physical, interpersonal activities. Pharmacological therapy for Cannabinoid deficiency is widely available in the form of medical and recreational cannabis and marijuana. Step-wise reduction of pharmacological therapy can be indicated to eliminate negative impact on long-term brain network structure. All other anti-depressants and stimulants are strictly contraindicated as they may drive self-focus and self-esteem (e.g., SSRIs) or competitiveness (Stimulants) counterproductive for therapy success. Monitoring can be focused on increased brain activity in brain regions associated with trust and social interaction detected by fMRI and increasing the number of actual memories with an above-average rating in attachment love and amusement. Structural MRI focusing on increase of rGMV in rIC and rPC is the most critical monitoring indicator for patients in therapy for a Cannabinoid-impairment.

In one or more examples, an exemplary treatment plan for opioid deficiency is presented below. In one or more examples, as a central output of activities with positive emotions, a hallmark of an opioid deficiency can a full set of peak-memories, however with a lower number or intensity rating in gratitude and pleasure. A key differentiating factor can be that even peak memories are deficient in number and intensity. Actual memories can show an overall lower rating on emotional significance and a below-average rating for gratitude and pleasure. In one or more examples, among individuals with an opioid gap, acute or historical substance use or abuse is abundant and focused towards THC-containing drugs, psychedelics, ketamine, anxiolytics and significantly less focused on stimulants, such as amphetamine (e.g., ADDERALL), methylphenidate (e.g., RITALIN), cocaine or methamphetamine. In parallel, a high use of medical pain killers, such as NSAIDS can be observed and should be seen as a differentiating factor. Acute or historical behavioral abuse is primarily focused around passive movie and television watching, e.g. binge-watching.

In terms of brain scan data, structural MRI (sMRI) can show significantly lower rGMV in the rIC and rPC compared to controls. In one or more examples, in response to past peak-memories, functional MRI (fMRI) can show activity in all brain regions carrying value-signals, such as right-anterior medial prefrontal cortex (mPFC), right-dorsal mPFC, right mPFC, left-dorsal PFC, left mPFC, left-anterior PFC and anterior cingulate cortex. In contrast, in response to actual memories signal intensity can be significantly reduced across all these areas. The number of peak memories that were recorded under the support of stimulating drugs can provide an indication on the respective opioid gap (compare fMRI intensity of drug-supported peak memories versus non-drug-supported peak memories).

In one or more examples, core to therapy for opioid impairment is behavioral therapy. It one or more examples, behavioral therapy can be focused on the experience of gratitude over pleasure. This can be most effectively achieved with yoga, meditation, mindfulness-coaching/training, and activities alone in nature. In one or more examples, group activities or activities of competitive nature may be contraindicated. In one or more examples, pharmacological therapy within the Opioid class can be limited due to high risk of substance addiction. If needed, NSAIDS can be indicated for an interim period to address any pain-related issues. In one or more examples, anti-depressants targeting the serotonin reward system only, such as provide a certain efficacy and can be used. Medical ketamine, THC and substances stimulating the CB1/2 systems can be used as well. In contrast, antidepressants also targeting the dopamine pathway as well as any stimulants can be strictly contraindicated. Over time, pharmacological therapy should be reduced and discontinued. During therapy, monitoring can focus on the increase of fMRI intensity for actual memories and the number increase of peak memories created without stimulating drug use. Structural MRI focusing on increase of rGMV in rIC and rPC can be a critical monitoring indicator for patients in therapy for an opioid-impairment.

As demonstrated above, therapies to counter-act reward molecule deficiencies can be highly specific to the precise reward system molecule that is found to be deficient. Thus, returning to the example of FIG. 4, the treatment plan can be selected be selected based on the identified deficiency found in the information received at step 402. In one or more examples, once the treatment plan has been selected at step 404, in one or more examples, the process 400 can move to step 406 wherein the treatment plan is applied to the patient. In one or more examples, applying the treatment plan to the patient can include engaging the patient in both behavioral and/or pharmacological therapies. In one or more examples, once the therapies have been applied to the patient at step 406, the progress of the patient can be monitored at step 408 to determine the effectiveness of the treatment.

In one or more examples, monitoring the progress of the treatment can include monitoring on a daily basis and assessing positive memories and emotions and observing derived reward system information. Additionally or alternatively, objective changes in brain structure and functionality by fMRI and EEG imaging can be monitored after three months and if needed after six months again to confirm tangible, physiological improvements in the brain.

In one or more examples, one or more studies can be conducted that will confirm the effectiveness of the methods described above with respect to FIGS. 1-4. For instance, in one or more examples a study can be conducted that can prove effectiveness of fMRI-guided individual behavioral actions on improvement of wellbeing and reduction of depression symptoms. In one or more examples, the study can include conducting an fMRI or other brain scan on a plurality of subjects and then generating a treatment plan for the patient similar to the process 400 described with respect to FIG. 4. In one or more examples, and prior to the treatment plan being applied the patient can be assessed using one or more questionnaires or clinical depression assessments. In one or more examples, after the reward molecule deficiency is identified according to the exemplary process 300 of FIG. 3, and a treatment plan is selected and administered according to the example of process 400 of FIG. 4, then the participants in the studying after a predetermined amount of time has passed (for instance 3 months) can have their changes in well-being as manifested in biomarkers (blood biomarkers), structural brain data (sMRI, EEG), functional brain data (fMRI, EEG upon memory recall) and wellbeing data (HAMD17, SWB) recorded to determine if any improvements to well-being can be observed. In one or more examples, the above study can detect improvement of HAMD17 and SWB score by individual behavioral actions guided by personal memory/emotion data. Additionally or alternatively, the study described above can assess correlation of biomarker and/or brain imaging data with HAMD17 and SWB score improvement. Additionally or alternatively, the study described above can assess correlation post-hoc memory recall) of fMRI data to predicted reward-emotion-map.

In one or more examples, another exemplary study that will validate the methods described above can include an interventional study in patients with mild/moderate depressive disorder before first-line pharmacological intervention. In one or more examples, a comparison of standard of care (treatment with an anti-depressant, no diagnostics) versus personalized pharmacological and behavioral intervention predicted by fMRI-guided reward-emotion-map as described above can be conducted. In one or more examples, the study can be configured to prove the effectiveness of fMRI-guided individual pharmacological and behavioral therapy on improvement of depression symptoms over the conventional standard-of-care. In one or more examples, participants can include individuals who have scored between 10-17 on the HAMDD17 assessments. In one or more examples, the participants in the study can all be assessed using the methods described above to determine one or more reward molecules deficiencies. However, in terms of treatment, a blind and randomized group of participants can receive a custom treatment plan (both behavioral and pharmacological) based on the identified deficiency, while another group can receive care according to the conventional standard of care.

In one or more examples, the two group's outcomes can be assessed by looking to determine if there was any significant improvement of HAMD17 and SWB in patients treated with individual therapy over standard of care across all treatment groups. Additionally, the correlation between biomarker and/or brain imaging data with HAMD17 and SWB score improvement can be assessed.

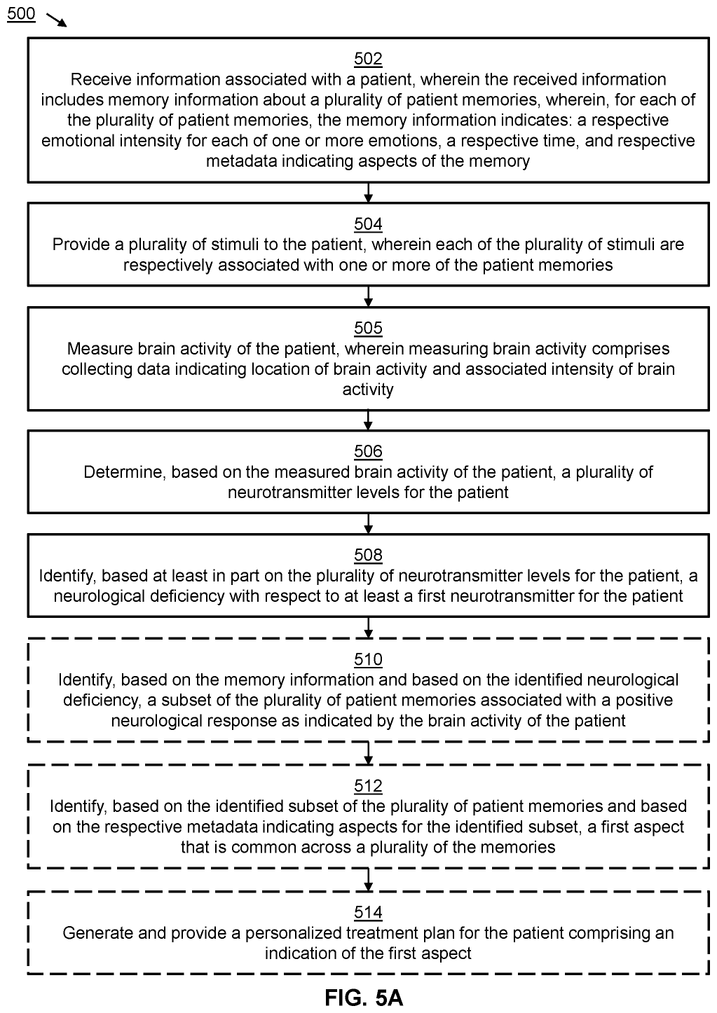

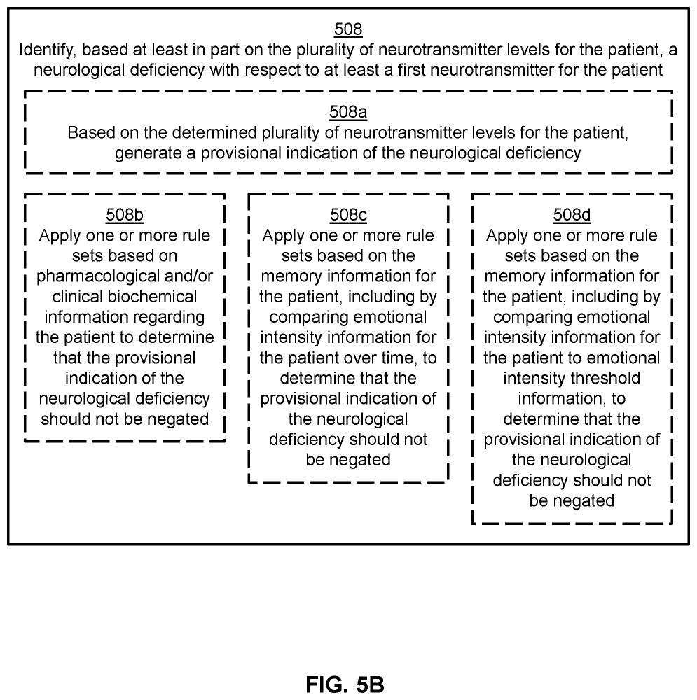

FIGS. 5A-B illustrate an exemplary method 500 for identifying and treating a neurological deficiency in a patient (or in any human subject), according to some aspects of the present disclosure. Method 500 may be performed, in whole or in part, automatically by a computerized system including one or more processors.

At step 502, the system may receive information associated with a patient, including memory information about a plurality of the patient's memories. The patient may input information into the system relating to a plurality of memories and/or experiences, including memories from the distant past and/or memories from the recent past (e.g., daily experiences). In some embodiments, the patient may input information regarding positive memories only. In some embodiments, one or more of the memories may be peak-positive-memories-memories that elicited a strong positive emotional response and/or that are quantitatively qualified by the patient as one of the best memories of the patient's life. For each of the plurality of patient memories, the memory information provided by the patient (e.g., by providing one or more inputs into a graphical user interface configured to collect information for analysis by the system) may include a respective emotional intensity for each of one or more emotions. The one or more emotions can include, but are not limited to, enthusiasm, sexual desire, pride/recognition, nurturant love, contentment, attachment love, amusement, pleasure, and gratitude. The emotional intensity data can be, for example, on a scale of 0-8, with 0 representing the patient reported not experiencing the respective emotion at all, and with 8 representing that the patient reported experiencing the respective emotion at the highest intensity.

For each of the plurality of patient memories, the memory information may include respective time information. The respective time information may include the duration of the emotions felt during the memory. The respective time information may include the date and/or time that the memory occurred. The respective time information may include date and/or time that the memory information was recorded by the system.

For each of the plurality of patient memories, the memory information may include respective metadata indicating aspects of the memory. Aspects of the memory may include, but are not limited to, information regarding a location associated with the memory, people associated with the memory, or activities associated with the memory. In some embodiments, content of a memory may be “tagged” with metadata labels indicating a person, group, place, and/or activity such that memories relating to overlapping or similar content may be grouped together by common tags.

For each of the plurality of patient memories, the memory information may also include representative media (e.g., audio/visual media such as photos, music, and/or videos) associated with the memory.

The system may store information in a database representing an association between each respective memory and the other collected information associated with the memory, including but not limited to emotions and associated ratings, time information, aspects of the memory (e.g., content of the memory), and/or media associated with the memory.

At step 504, the system may provide a plurality of stimuli to the patient. Each of the plurality of stimuli may be associated with one or more of the patient memories. For example, after collecting memory information from a patient, stimuli selected based on the memory information provided may be provided to the patient. In some embodiments, the plurality of stimuli may include a randomized (or semi-randomized) collection of photos, audio, and/or videos that include media information associated with one or more of the provided patient memories. When presented to the patient, the media information associated with the patient's memories may be mixed in with other random images, audio, and/or video, which may act as a control. Control may also be provided by presenting the patient with noise and/or blank images. In some embodiments, media may be presented in predetermined time windows, for example by presenting the patient with the plurality of stimuli may be alternated with control stimuli. Control stimuli may include, in some embodiments, one or more of the following: photographs and/or video that are not known to be associated with any specific emotional response for the patient. Such a photo may be combined with white noise. After each stimuli, control or emotional memory, there may be a re-baselining phase when the patient is presented with a black screen with a center-cross in order to “wash-out” all emotions before the next stimuli is shown. Emotional stimuli may be randomly alternated with control stimuli. After each stimuli (control or emotional), the patient may be reset to emotional an baseline before the next stimuli (e.g., by presenting a black screen).

At step 505, the system may measure the brain activity of the patient. Brain activity of the patient may be measured while stimuli (including memory-related stimuli and control stimuli) are presented to the patient. Step 505 may be performed concurrently with step 504, such that brain activity is measured as various stimuli from the plurality of stimuli are provided to the patient. Measuring brain activity may be done by performing structural magnetic resonance imaging (sMRI), functional magnetic resonance imaging (fMRI), real-time functional magnetic resonance imaging (rt-fMRI), real-time functional magnetic resonance imaging neurofeedback (rt-fMRI-NF), a full-scalp electroencephalogram (EEG), and/or full-scalp functional near-infrared spectroscopy (fNIRS). The rt-fMRI scan may be a 3 Tesla rt-fMRI scan or a 7 Tesla rt-fMRI scan. Measuring brain activity may include measuring the intensity of brain activity while one or more stimuli are provided to the patient, for example in the form of audio, image, and/or video. Measuring brain activity may also include recording the location of the brain activity while one or more stimuli are provided to the patient. The location of the brain activity may indicate a three-dimensional location at which a signal (including a respective signal intensity) representing brain activity is measured.

At step 506, the system may determine a plurality of neurotransmitter levels for the patient based on the collected data indicating the location of brain activity and associated intensity of brain activity. The plurality of neurotransmitters may include, but is not limited to, dopamine, testosterone, serotonin, oxytocin, cannabinoids, and/or opioids. The system may use the three-dimensional location of the brain activity and associated intensity to identify activity in pre-defined reward centers in the brain that can be correlated with certain emotions and with certain neurotransmitters. For example, brain activity in a certain area of the brain may be known to correspond to one or more emotions and/or neurotransmitters, for example based on background knowledge developed based on testing of a specific patient's brain activity and/or based on testing of brain activity from a population of subjects. The system may then detect activity at that certain area of the brain, and may determine based on the detected activity at that area that the patient is experiencing activity of the associated neurotransmitters (and/or associated emotions). The intensity of signal data measured at the area may be used to determine an intensity of a neurotransmitter response (and/or an intensity of emotional experience) for example based on a positive correspondence between signal intensity data and intensity of neurotransmitter response.

In some embodiments, the system may use an algorithm to determine which neurotransmitters are associated with the one or more emotions indicated in the memory information. For example, the system may use the PE-NT matrix of FIG. 2. For example, by using the PE-NT matrix, dopamine may be associated with enthusiasm, sexual desire, pride/recognition, nurturant love, contentment, amusement, attachment love, pleasure, or gratitude. Testosterone may be associated with sexual desire. Serotonin may be associated with pride/recognition. Oxytocin may be associated with nurturant love or contentment. Cannabinoids may be associated with contentment, amusement, or attachment love. Opioids may be associated with amusement, pleasure, or gratitude.

In some embodiments, the system may aggregate brain scan data by grouping the brain scan data according to common neurotransmitters and/or common emotions. For example, brain scan data collected during presentation of stimuli associated with memories that are tagged with a certain emotion may be analyzed collectively, for example by analyzing a total signal intensity and/or average signal intensity measured during presentation of all of said memories.

In some embodiments, brain scan data may be grouped according to memories that are associated with the same emotion (e.g., based on subjective emotional experience information provided by the user).

In some embodiments, brain scan data may be grouped according to memories that are associated with different emotions but wherein the different emotions are associated with the same neurotransmitter. For example, to obtain aggregated brain activity data associated with dopamine, the system may aggregate all the brain activity data measured as a response to stimuli meant to evoke enthusiasm. To obtain aggregated brain activity associated with serotonin, the system may aggregate brain activity data measured as a response to stimuli meant to evoke pride/recognition.

The system may aggregate the brain activity data associated with a specific neurotransmitter in a way such that it minimizes the effect of other neurotransmitters on the brain activity data. For example, even though dopamine is associated with a plurality of emotions, in some embodiments, the system may only aggregate brain data measured as a response to stimuli meant to evoke enthusiasm because dopamine is the only neurotransmitter (of the group of dopamine, testosterone, serotonin, oxytocin, cannabinoids, and opioids) associated with enthusiasm. In one or more examples, the system may process the aggregated brain data associated with dopamine to filter out the effect of other neurotransmitters on the aggregated brain data, for example to isolate a brain activity signal response attributable to dopamine activity.

The resulting aggregated brain activity data can be used to detect activity (or lack thereof) in pre-determined reward centers of the brain. Based on the signal intensity in these pre-determined reward centers of the brain, the system may determine a plurality of neurotransmitters. In one or more examples, determining the plurality of neurotransmitters may be determined by comparing the signal intensity in a pre-determined reward center to a threshold signal intensity level for that pre-determined reward center. The threshold level may be determined based on a historical analysis of a specific patient and/or based on a historical analysis of a population of subjects. For example, the system can analyze the aggregated brain activity data associated with dopamine to determine whether it indicates activity in the pre-determined reward center of the brain associated with dopamine. If there is any activity in the pre-determined reward centers of the brain associated with dopamine, the system can determine the signal intensity in these locations. Based on the determined signal intensity in the pre-determined reward centers associated with dopamine, the system can determine a level of dopamine. The system may compare the determined signal intensity in the pre-determined reward centers associated with dopamine to a threshold signal intensity for those reward centers to determine a level of dopamine.

Regions of interest in the brain correlated with emotions and neurotransmitters can be determined by employing a brain study. The brain study can be configured to map emotions and reward system activity while the brain is experiencing memory recall to create a predictive map of the landscape for emotions (and for corresponding reward center activity and/or neurotransmitter activity) within the brain. In one or more examples, a brain study can be performed on each individual patient to create a predictive map of the individual patient's landscape for emotions and neurotransmitters. In one or more examples, a brain study can be conducted on multiple control subjects, which may in some embodiments include control subjects who are not known to have any neurotransmitter deficiency or who are affirmatively believed to not have any neurotransmitter deficiency.

In one or more examples, the brain study is performed on subjects who have a fully prepared memory and emotion database (memories associated with positive emotions) that can include a plurality of person positive memories and can have one or more peak memories (e.g., memories that elicited the greatest emotional response). The brain study can include providing a plurality of stimuli associated with the memories and pre-defined emotions while taking a brain scan of the patient. The brain scans can be analyzed to determine distinct signal patterns, e.g., activity in certain regions of interest in the brain, and those regions of the brain may thereby be correlated to the pre-defined emotions. The determined distinct signal patterns can thus be used to determine regions of interest in the brain that are associated with specific emotions. The system can then use an algorithm, such as the PE-NT matrix of FIG. 2, to determine which regions of interest, now known to be associated with one or more specific emotions, are associated with specific reward systems and/or neurotransmitters. For example, if a patient is exposed to a stimulus that has been pre-defined (e.g., by labeling by the patient) as eliciting an enthusiasm reaction, the system can determine that the activated brain region is a region of interest associated with enthusiasm, and the system can therefore further determine (e.g., using a PE-NT matrix) that the region of interest is associated with the neurotransmitter Dopamine.

In some embodiments, the system may employ one or more alternative or additional techniques for determining regions of interest in the brain that are determined to be associated with certain emotions and/or with activity of certain neurotransmitters. As described above, one technique involves measuring brain activity and identifying active regions of interest in the brain that are triggered when stimuli associated with certain emotions are presented, thereby creating a biomarker brain map that associates a brain region with an emotion. As noted above, the brain region known to be associated with the given emotion may then be linked to one or more associated neurotransmitters using an algorithm such as a PE-NT matrix. However, other techniques may additionally or alternatively be applied to link the brain regions to one or more associated neurotransmitters.

In one additional or alternative approach, one or more known pharmacological substances that either enhance or block one or more certain neurotransmitter pathways may be administered to subjects/patients, and the brain activity of the subjects/patients may then be measured under stimuli (e.g., the same stimuli as used before without pharmacological intervention) configured to elicit certain emotional responses. Differences may be measured in activity levels in one or more regions of interest in the brain as compared between (a) a state when the patient/subject has not been exposed to a pharmacological intervention and (b) a state when the patient/subject has been exposed to a pharmacological intervention. Regions of interest in the brain where statistically significant differences in activity level are observed may thus be determined to be associated with the neurotransmitters for which the pharmacological intervention is known to block.

Using any one or more of the approaches explained above, a molecular fingerprint may thus be developed for regions in the brain that are associated with given emotions and/or with given neurotransmitters.

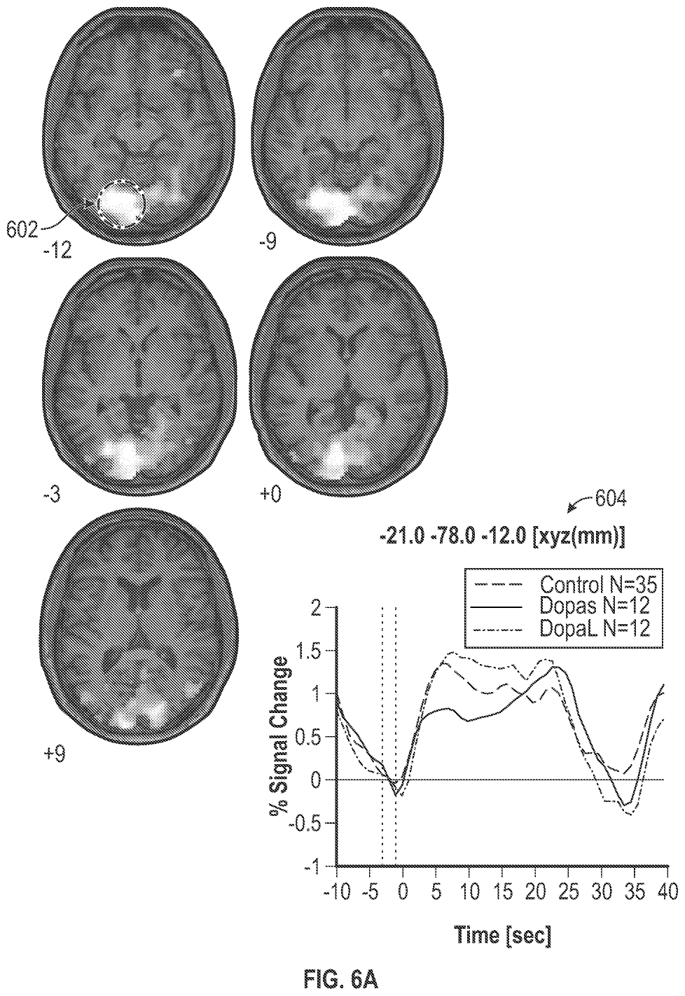

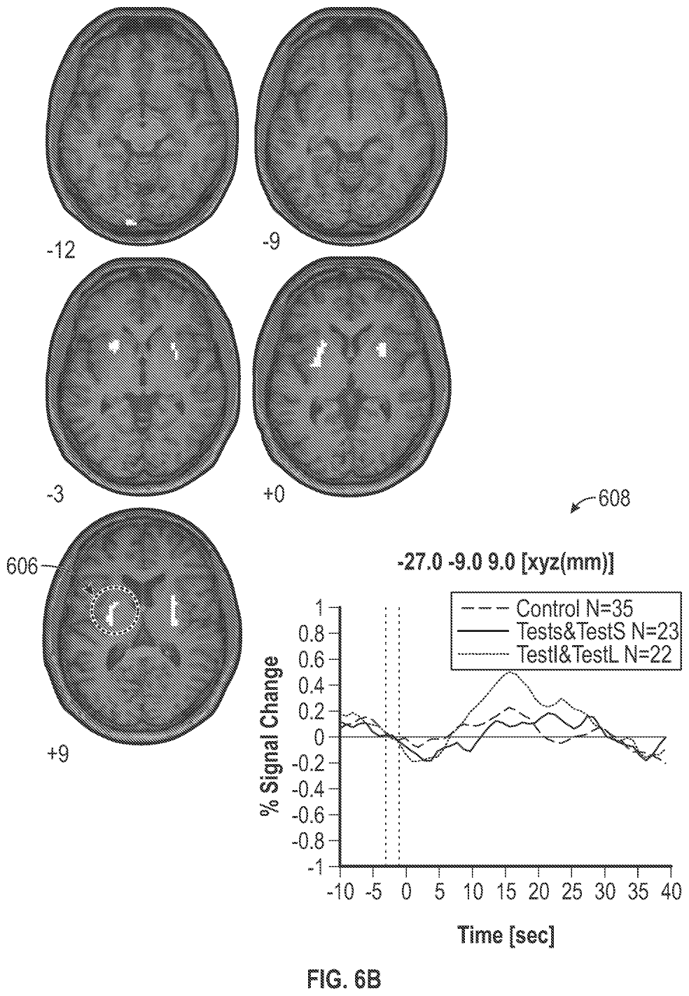

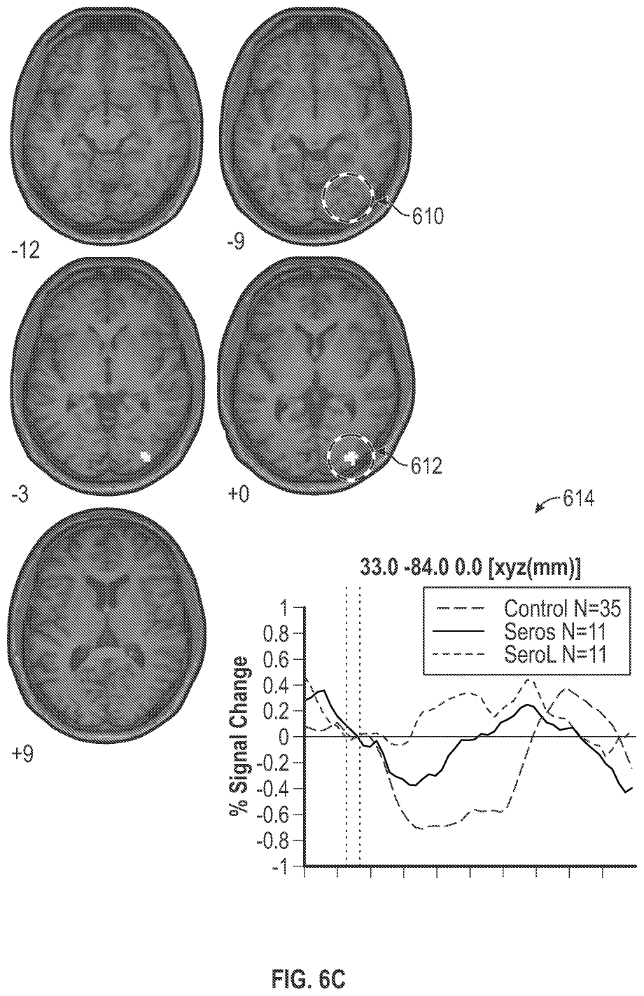

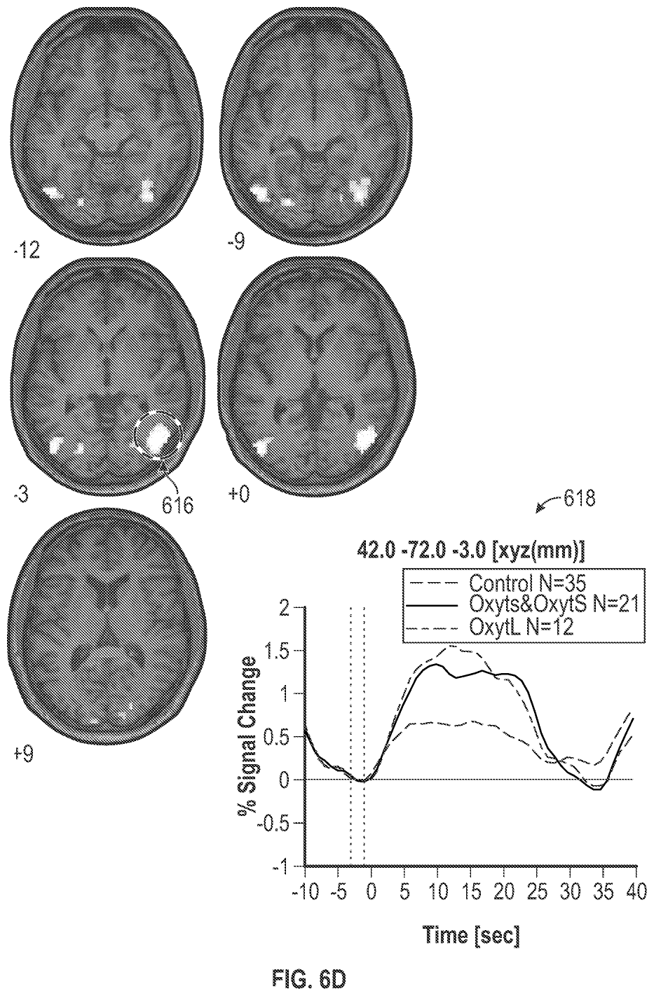

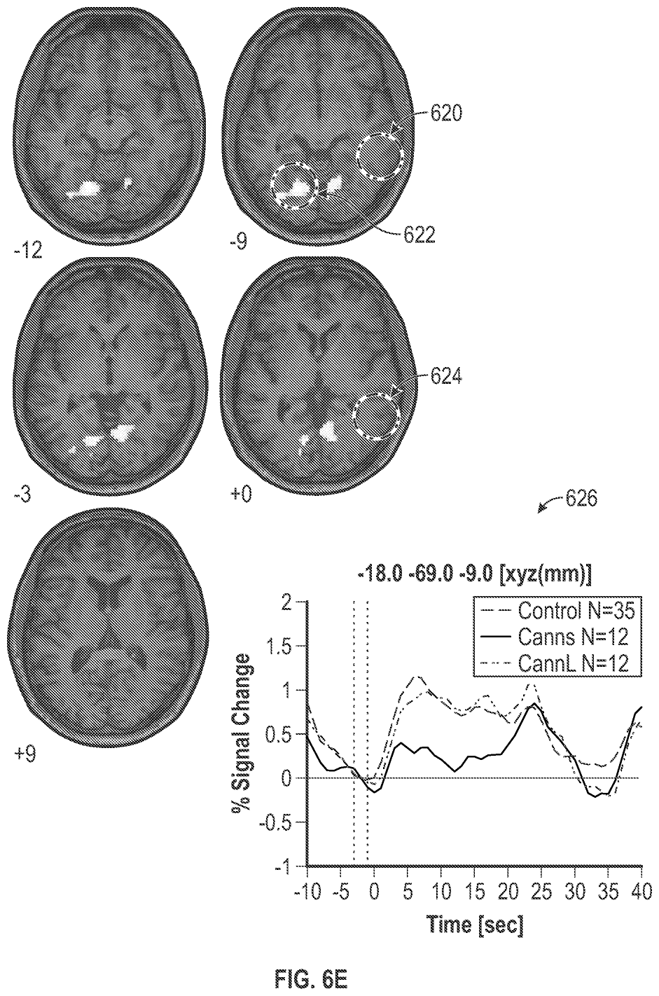

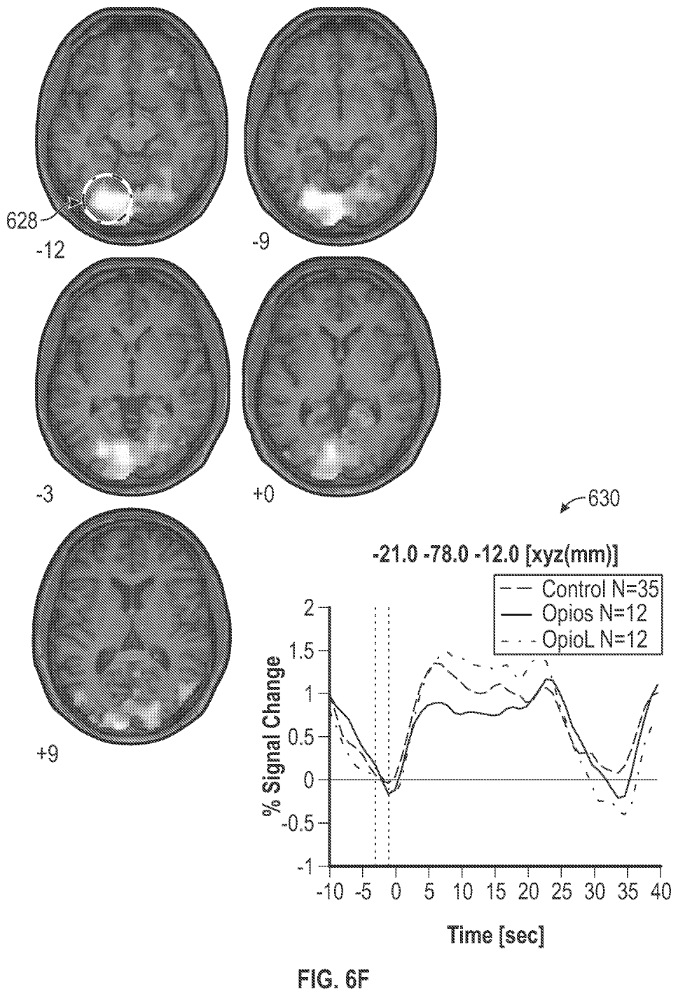

FIGS. 6A-6F illustrate exemplary aggregated brain activity data, wherein the brain activity is concentrated in regions of interest that are correlated with various emotions, reward centers, and/or neurotransmitters. FIG. 6A illustrates aggregated brain activity in regions of interest associated with enthusiasm and correlated with dopamine. Region 602 indicates a region of interest that is associated with enthusiasm (and thereby, associated with dopamine). The regions in FIG. 6A with brightened color indicate the location and intensity of the brain activity. Since the brain activity overlaps with region 602, and the brightness indicates a high intensity of brain activity in region 602, the system calculates a high level of dopamine. Line-graph 604 indicates percent signal change with respect to time for the control stimuli, stimuli not associated with dopamine (e.g., stimuli not associated with enthusiasm or rated low for enthusiasm, such as stimuli associated instead more strongly with other neurotransmitters), and stimuli associated with dopamine. In the example shown, brain activity was measured for the stimuli (or for the control) over a 20-second period and brain activity was measured over that period. Line-graph 604 indicates that the difference between brain activity associated with dopamine and brain activity not associated with dopamine is statistically significant.

FIG. 6B illustrates aggregated brain activity in regions of interest associated with sexual desire and correlated with testosterone. Region 606 indicates a region of interest that is associated with sexual desire (and thereby, associated with testosterone). The regions in FIG. 6B with brightened color indicate the location and intensity of the brain activity. Since the brain activity overlaps with region 606, but the brightened area indicates a low intensity of brain activity in region 606, the system calculates a low level of testosterone. Line-graph 608 indicates percent signal change with respect to time for the control stimuli, stimuli not associated with testosterone (e.g., stimuli not associated with sexual desire or rated low for sexual desire, such as stimuli associated instead more strongly with other neurotransmitters), and stimuli associated with testosterone. Line-graph 608 indicates that the difference between brain activity associated with testosterone and brain activity not associated with testosterone is statistically significant.

FIG. 6C illustrates aggregated brain activity in regions of interest associated with pride/recognition and correlated with serotonin. Regions 610 and 612 indicate regions of interest that are associated with pride/recognition (and thereby, associated with serotonin.) The regions in FIG. 6C with brightened color indicate the location and intensity of the brain activity. Since the brain activity overlaps with region 612, the brightness indicates a low intensity of brain activity in region 612, and no brain activity overlaps with region 610, the system calculates a negligible level of serotonin. Line-graph 614 indicates percent signal change with respect to time for the control stimuli, stimuli not associated with serotonin (e.g., stimuli not associated with pride/recognition or rated low for pride/recognition, such as stimuli associated instead more strongly with other neurotransmitters), and stimuli associated with serotonin. Line-graph 614 indicates that the difference between brain activity associated with serotonin and brain activity not associated with serotonin is statistically significant.

FIG. 6D illustrates aggregated brain activity in regions of interest associated with nurturant love and contentment and correlated with oxytocin. Region 616 indicates a region of interest that is associated with nurturant love and contentment (and thereby, associated with oxytocin). The regions in FIG. 6D with brightened color indicate the location and intensity of the brain activity. Since the brain activity overlaps with region 616 and the brightness indicates a high intensity of brain activity in region 616, the system calculates a high level of oxytocin. Line-graph 618 indicates percent signal change with respect to time for the control stimuli, stimuli not associated with oxytocin (e.g., stimuli not associated with nurturant love or rated low for nurturant love, such as stimuli associated instead more strongly with other neurotransmitters), and stimuli associated with oxytocin. Line-graph 618 indicates that the difference between brain activity associated with oxytocin and brain activity not associated with oxytocin is statistically significant.

FIG. 6E illustrates aggregated brain activity in regions of interest associated with attachment love and amusement, associated to a lesser extent with contentment, and correlated with cannabinoids. Regions 620, 622, and 624 indicate regions of interest that are associated with attachment love, amusement, and contentment (and thereby, associated with cannabinoids). The regions in FIG. 6E with brightened color indicate the location and intensity of the brain activity. Since the brain activity overlaps with region 622, the brightness indicates a medium intensity of brain activity in region 622, and no brain activity is located in regions 620 and 624, the system calculates a negligible level of cannabinoids. Line-graph 626 indicates percent signal change with respect to time for the control stimuli, stimuli not associated with cannabinoids (e.g., stimuli not associated with attachment love or rated low for attachment love, such as stimuli associated instead more strongly with other neurotransmitters), and stimuli associated with cannabinoids. Line-graph 626 indicates that the difference between brain activity associated with cannabinoids and brain activity not associated with cannabinoids is statistically significant.

FIG. 6F illustrates aggregated brain activity in regions of interest associated with pleasure and gratitude and correlated with opioids. Region 628 indicates a region of interest associated with pleasure and gratitude (and thereby, associated with opioids). The regions in FIG. 6F with brightened color indicate the location and intensity of the brain activity. Since the brain activity is overlaps with region 628 and the brightness indicates a high intensity of brain activity in region 628, the system calculates a high level of opioids. Line-graph 630 indicates percent signal change with respect to time for the control stimuli, stimuli not associated with opioids (e.g., stimuli not associated with or rated low for pleasure and/or gratitude), and stimuli associated with opioids. Line-graph 630 indicates that the difference between brain activity associated with opioids and brain activity not associated with opioids is statistically significant.

Returning to FIGS. 5A-5B, at step 508, the system can identify neurological deficiencies of the patient with respect to neurotransmitters based at least in part on the plurality of neurotransmitter levels calculated in step 506. In some embodiments, the system may use only the plurality of neurotransmitter levels calculated in step 506 to determine neurological deficiencies. For example, using the neurotransmitter levels obtained from the depicted example in FIGS. 6A-6F, the system may identify the neurological deficiencies as shown below in Table 1.

TABLE 1
Example Identification of Neurological Deficiency Based on Neurotransmitter Levels

| | Dopamine | Testosterone | Serotonin | Oxytocin | Cannabinoids | Opioids |
|---|---|---|---|---|---|---|
| Neurotransmitter | High | Low | Negligible | High | Negligible | High |
| levels from brain | | | | | | |
| activity data | | | | | | |
| Deficiency | No | Yes | Yes | No | Yes | No |

Neurotransmitter levels, including quantification of levels and/or classification of levels (e.g., negligible, low, moderate, high, etc.) may be compared to one or more target values, expected values, historical values, and/or threshold values in order to determine whether a neurological deficiency for the respective neurotransmitter is present.

Optionally, at step 508a, the system may use the plurality of neurotransmitter levels calculated in step 506 to generate a provisional indication of neurological deficiencies. Optionally, at step 508b, the system may apply one or more rule sets based on pharmacological and/or clinical biochemical information regarding the patient to determine whether the provisional indication of a neurological deficiency should be negated. For example, if the provisional indication of neurological deficiency shows a testosterone deficiency, information regarding the patient's blood testosterone levels can be used to negate or validate the provisional indication of a testosterone deficiency. In another example, information regarding a history of ineffective Selective Serotonin Reuptake Inhibitor (SSRI) treatment can be used to negate the provisional indication of a serotonin deficiency.

Optionally at steps 508c and 508d, the system may apply one or more rule sets based on the memory information, to determine that the provisional indication of the neurological deficiency should not be negated. Using the one or more rule sets, the system may analyze the memory information.

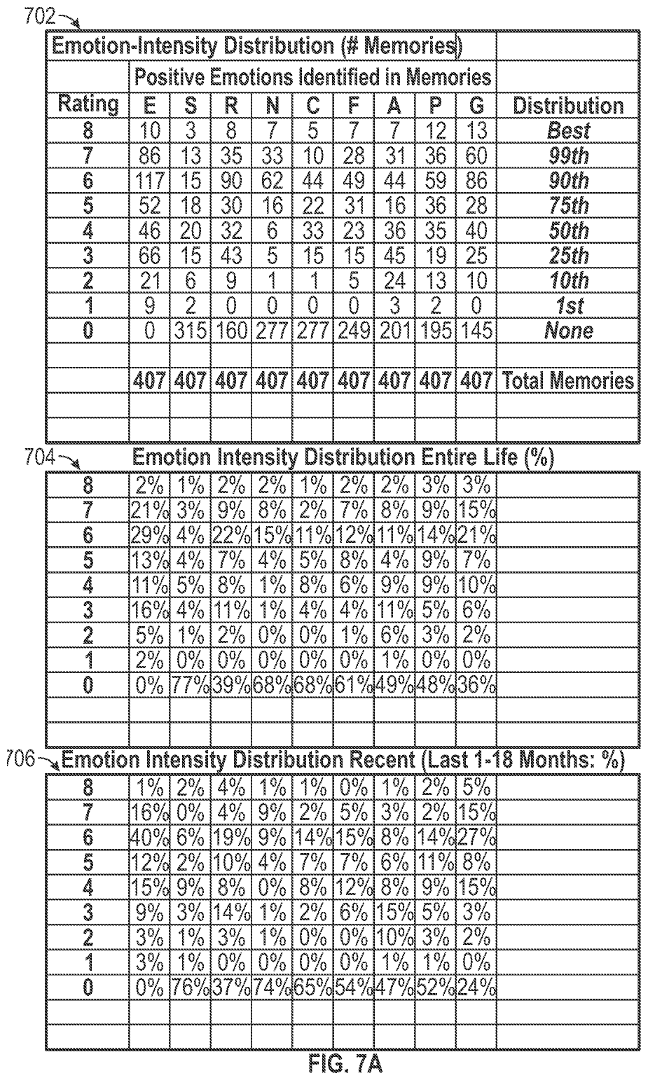

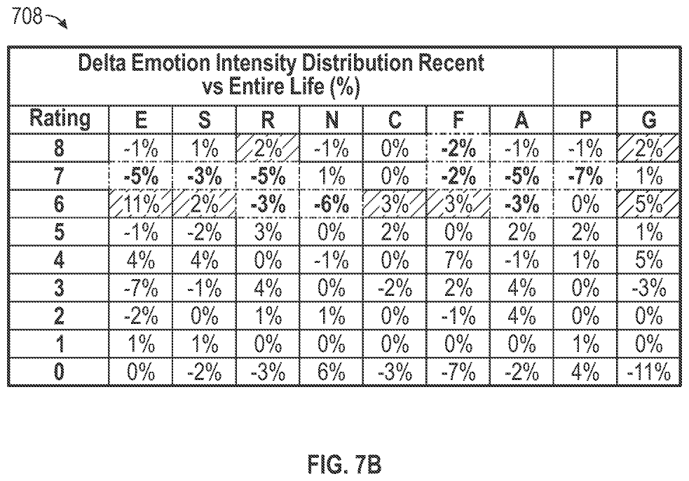

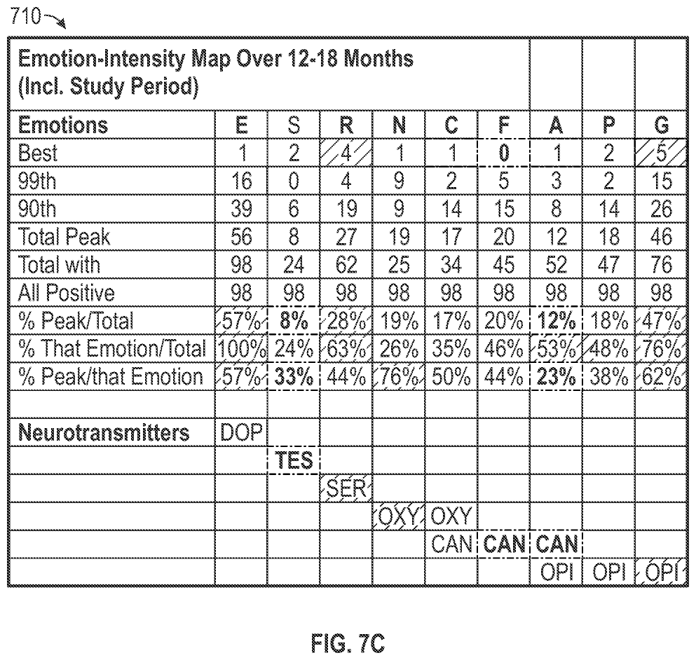

FIGS. 7A-C illustrates an exemplary analysis performed on the memory information. With respect to FIG. 7A, the system may aggregate the memory information into emotion-intensity distribution 702, which counts the number of memories for each rating of each emotion. In the depicted example, emotion-intensity distribution 702 indicates that the memory information included ten memories where enthusiasm had a rating of 8. The system may use the emotion-intensity distribution 702 to calculate the percentage of memories in each emotional rating category for the patient's entire life, as seen in chart 704. The system may use the emotion-intensity distribution 702 to calculate the percentage of memories in each emotional rating category only using memories from the most recent twelve to eighteen months, as seen in chart 706.

Referring back to FIG. 5A, at step 508c, the system may compare emotional intensity information for the patient over time to determine that the provisional indication of the neurological deficiency should not be negated. For example, with reference to FIG. 7B, the system may compare the percentage of memories in each emotional rating category from the patient's entire life (e.g., chart 704 in FIG. 7A) to the percentage of memories in each emotional rating category from the last twelve to eighteen months of the patient's life (e.g., chart 706 in FIG. 7A) to obtain a delta emotion-intensity distribution, as seen in delta emotional intensity distribution 708. The system may use this information to validate the provisional indication of the neurological deficiency. For example, delta emotional intensity distribution 708 indicates that the patient has fewer memories with high a familial/attachment love rating over time, validating the provisional indication of a cannabinoid deficiency.

Referring back to FIG. 5A, at step 508d, the system may compare emotional intensity information for the patient to emotional intensity threshold information, to determine that the provisional indication of the neurological deficiency should not be negated. For example, with reference to FIG. 7C, the system may use the percentage of memories in each emotional rating category from the last twelve to eighteen months of the patient's life (e.g., chart 706 in FIG. 7A) to create an emotion-intensity map, as seen in emotion-intensity map 710. The emotion-intensity map may include calculations for the percentage of peak memories over total memories for each emotion. The system may apply one or more threshold-based analyses or other data-processing analyses to the emotion-intensity map to determine a neurological deficiency, and may then use that determination to validate the provisional indication of the neurological deficiency that was determined based on the brain scan data. In the depicted example, the percentage of peak amusement memories over total memories falls below the threshold, indicating a cannabinoid deficiency, thereby validating a provisional indication of cannabinoid deficiency.

The resulting neurological deficiencies (which may be output as a final diagnosis of a deficiency and/or as a provisional diagnosis of deficiency) from the depicted example following step 508d, are summarized below in Table 2.

TABLE 2
Example Identification of Neurological Deficiency
Based on Brain Activity and Memory Data

| | Dopamine | Testosterone | Serotonin | Oxytocin | Cannabinoids | Opioids |
|---|---|---|---|---|---|---|
| Neurotransmitter | High | Low | Negligible | High | Negligible | High |
| levels from brain | | | | | | |
| activity data | | | | | | |
| Provisional | No | Yes | Yes | No | Yes | No |
| Indication of | | | | | | |
| Deficiency | | | | | | |
| Indication of | No | Yes | No | No | Yes | No |
| Deficiency from | | | | | | |
| Emotion- | | | | | | |
| Intensity Map | | | | | | |
| Deficiency | No | Yes | Yes | No | Yes | No |

In some examples, the system may perform some or all of steps 508a-508d. The resulting neurological deficiencies from the depicted example obtained by following steps 508a-508c, are summarized below in Table 3.

TABLE 3
Example Identification of Neurological Deficiency Based on Brain Activity,
Pharmacological Data, Clinical Biochemical Data, and Memory Data

| | Dopamine | Testosterone | Serotonin | Oxytocin | Cannabinoids | Opioids |
|---|---|---|---|---|---|---|
| Neurotransmitter | High | Low | Negligible | High | Negligible | High |
| levels from brain | | | | | | |
| activity data | | | | | | |
| Provisional | No | Yes | Yes | No | Yes | No |
| Indication of | | | | | | |
| Deficiency | | | | | | |
| Clinical | N/a | No (healthy | N/a | N/a | N/a | N/a |
| Biochemistry | | levels) | | | | |
| Pharmacological | N/a | N/a | No (previous | N/a | N/a | N/a |
| Data | | | SSRI | | | |
| | | | treatment | | | |
| | | | ineffective) | | | |
| Indication of | No | Yes | No | No | Yes | No |
| Deficiency from | | | | | | |
| Emotion- | | | | | | |
| Intensity Map | | | | | | |
| Deficiency | No | No | No | No | Yes | No |

Referring back to FIG. 5A, the method may proceed to steps 510-514, which may be used to generate and provide a treatment plan for a patient based on an identified neurological deficiency, as explained below.

The system may optionally perform step 510 by identifying a subset of the plurality of patient memories associated with a positive neurological response based on the memory information and the identified neurological deficiency. For example, if system identified a cannabinoid deficiency, the system may identify all or some of the memories from the plurality of memories that demonstrated strong cannabinoid (and/or attachment love) responses when corresponding stimuli were presented to the patient. That is, even if the user's cannabinoid responses were generally lower than expected, those memories that did elicit a strong cannabinoid response may be identified based on brain scan data. The system may identify those top memories showing strong responses for a deficient neurotransmitter where the response is over a threshold signal intensity level, and/or may identify a predetermined number of top memories with the strongest responses for the deficient neurotransmitter.

Optionally, at step 512, the system may identify one or more aspects that are common across that identified subset of memories based on the respective metadata for that subset of memories. For example, if the metadata for the subset of strong cannabinoid (and/or attachment love) memories indicates that all (or most) of the memories are associated with a common person or persons (e.g., a spouse, friend, or family member), then the system would identify that person or persons as a common aspect of the selected strong cannabinoid memories.

Optionally, at step 514, the system may generate and provide a treatment plan for the patient that includes the identified aspect of the subset of memories. For example, if the identified aspect from step 512 was a certain person or group of people, system would generate a treatment plan configured to instruct the patient to spending more time with that person or group of people. In some embodiments, the treatment plan may include a data structure configured to provide instructions to a patient, provide instructions to a physician, and/or provide machine-executable instructions for a system configured to automatically implement the treatment plan. Automatic (e.g., automated or semi-automated) implementation of a treatment plan may include exposure to stimuli associated with a subject that elicits a strong positive response in a deficient neurotransmitter, such as providing media to the patient that is associated with the subject. In some embodiments, automatic implementation of the treatment plan may include automatically facilitating electronic communication between a patient (e.g., using a mobile application) and a subject (e.g., a person or activity) that elicits a strong positive response in a deficient neurotransmitter; for example, implementation of the treatment plan may automatically open a chat for a the cannabinoid-deficient patient to communicate with a family member who provides a strong cannabinoid response for the patient.

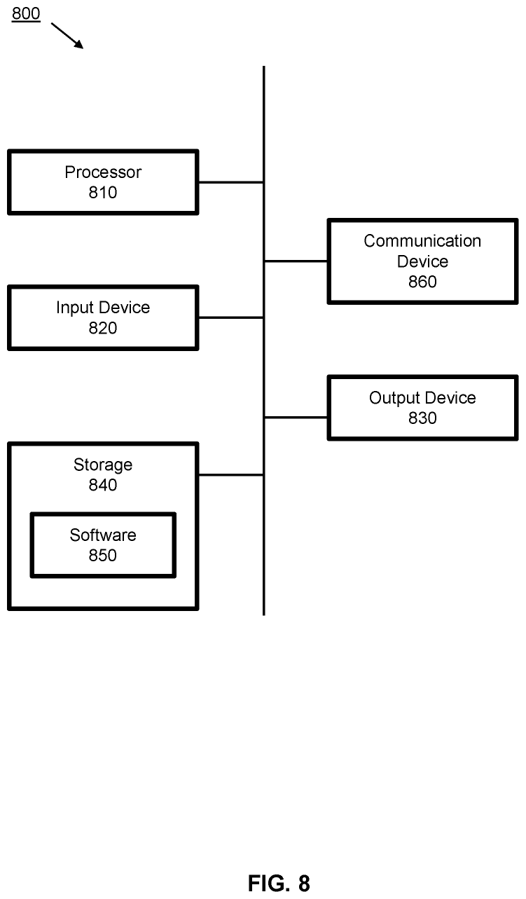

FIG. 8 illustrates an example of a computing device in accordance with one embodiment. Device 800 can be a host computer connected to a network. Device 800 can be a client computer or a server. As shown in FIG. 8, device 800 can be any suitable type of microprocessor-based device, such as a personal computer, workstation, server, or handheld computing device (portable electronic device) such as a phone or tablet. The device can include, for example, one or more of processor 810, input device 820, output device 830, storage 840, and communication device 860. Input device 820 and output device 830 can generally correspond to those described above and can either be connectable or integrated with the computer.

Input device 820 can be any suitable device that provides input, such as a touch screen, keyboard or keypad, mouse, or voice-recognition device. Output device 830 can be any suitable device that provides output, such as a touch screen, haptics device, or speaker.

Storage 840 can be any suitable device that provides storage, such as an electrical, magnetic, or optical memory, including a RAM, cache, hard drive, or removable storage disk. Communication device 860 can include any suitable device capable of transmitting and receiving signals over a network, such as a network interface chip or device. The components of the computer can be connected in any suitable manner, such as via a physical bus or wirelessly.

Software 850, which can be stored in storage 840 and executed by processor 810, can include, for example, the programming that embodies the functionality of the present disclosure (e.g., as embodied in the devices as described above).

Software 850 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 840, that can contain or store programming for use by or in connection with an instruction execution system, apparatus, or device.

Software 850 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 800 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 800 can implement any operating system suitable for operating on the network. Software 850 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

Following is a list of exemplary enumerated embodiments, which may be combined in whole or in part with one another and/or with any other disclosure herein:
1. A method for treating a neurological disorder, the method comprising:
   receiving information associated with a patient, wherein the received information includes information about one or more past events that the patient has engaged in and one or more emotions associated with the one or more events;
   determining one or more reward molecule levels of the patient based on the received information associated with the patient;
   receiving one or more scans of the patient's brain;
   determining one or more reward molecule levels of the patient based on the received one or more scans of the patient's brain; and
   determining one or more reward molecule deficiencies of the user based on the determined one or more reward molecule levels of the patient based on the received information associated with the patient and based on the determined one or more reward molecule levels of the patient based on the received one or more scans of the patient's brain.
2. The method of embodiment 1, wherein the method comprises selecting a predetermined treatment plan for the determined one or more reward molecule deficiencies based on an identity of each reward molecule deficiency,
3. The method of embodiment 2, wherein the identity of the reward molecule deficiency is selected from a group consisting of: dopamine, serotonin, testosterone, oxytocin, cannabinoids, opioids.
4. The method of any one of embodiments 1-3, wherein determining one or more reward molecule levels of the patient based on the received information associated with the patient comprises applying the received information associated with the patient to a positive emotion to a neurotransmitter (PE-NT) matrix.
5. The method of any one of embodiments 1-4, wherein the received one or more scans of the patient's brain comprise real-time functional magnetic resonance imaging (rt-fMRI) scans.
6. The method of any one of embodiments 1-5, wherein the received one or more scans of the patient's brain comprise electroencephalogram (EEG) scans.
7. The method of any one of embodiments 1-6, the received one or more brains scans are acquired using a method comprising;
   exposing the patient to one or more stimuli;
   determining one or more locations within the brain where a signal is generated in response to the stimuli; and
   determining a signal intensity associated with each location of the brain where a signal is generated in response to the stimuli.
8 The method of claim 7, wherein determining one or more reward molecule levels of the patient based on the received one or more scans of the patient's brain comprises:
   associating the one or more determined locations within the brain where a signal is generated in response to the stimuli to one or more types of reward molecules; and
   determining the one or more reward molecule levels of the patient based on the determine signal intensity associated each location of the brain where a signal is generated in response to the stimuli.
9. The method of any one of embodiments 1-8, wherein the one or more emotions associated with the one or more events received information associated with the patient of the information associated with the patient comprises an emotion from the group comprising: enthusiasm, sexual desire, pride/recognition, nurturant love, contentment, attachment love, amusement, pleasure, and gratitude.
10. The method of any one of embodiments 1-9, wherein the received information associated with a patient comprises the patient's responses to one or more questionnaires configured to diagnose depression in the patient.
11. A method for identifying and treating a neurological deficiency, the method performed by a system comprising one or more processors, the method comprising:
   receiving information associated with a patient, wherein the received information includes memory information about a plurality of patient memories, wherein, for each of the plurality of patient memories, the memory information indicates: a respective emotional intensity for each of one or more emotions, respective time information, and respective metadata indicating aspects of the memory;
   providing a plurality of stimuli to the patient, wherein each of the plurality of stimuli are respectively associated with one or more of the patient memories;
   while providing the stimulus to the patient, measuring brain activity of the patient, wherein measuring brain activity comprises collecting data indicating location of brain activity and associated intensity of brain activity;
   determining, based on the measured brain activity of the patient, a plurality of neurotransmitter levels for the patient;
   identifying, based at least in part on the determined plurality of neurotransmitter levels for the patient, a neurological deficiency with respect to at least a first neurotransmitter for the patient.
12. The method of claim 11, comprising:
   identifying, based on the memory information and based on the identified neurological deficiency, a subset of the plurality of patient memories associated with a positive neurological response as indicated by the brain activity of the patient;
   identifying, based on the identified subset of the plurality of patient memories and based on the respective metadata indicating aspects for the identified subset, a first aspect that is common across a plurality of the memories; and
   generating and providing a personalized treatment plan for the patient comprising an indication of the first aspect.
13. The method of any one of embodiments 11-12, wherein identifying the neurological deficiency comprises:
   based on the determined plurality of neurotransmitter levels for the patient, generating a provisional indication of the neurological deficiency;
   applying one or more rule sets based on pharmacological and/or clinical biochemical information regarding the patient to determine that the provisional indication of the neurological deficiency should not be negated.
14. The method of any one of embodiments 11-13, wherein identifying the neurological deficiency comprises:
   based on the determined plurality of neurotransmitter levels for the patient, generating a provisional indication of the neurological deficiency;
   applying one or more rule sets based on the memory information for the patient, including by comparing emotional intensity information for the patient to emotional intensity threshold information, to determine that the provisional indication of the neurological deficiency should not be negated.
15. The method of any one of embodiments 11-14, wherein identifying the neurological deficiency comprises:
   based on the determined plurality of neurotransmitter levels for the patient, generating a provisional indication of the neurological deficiency;
   applying one or more rule sets based on the memory information for the patient, including by analyzing changes in emotional intensity information for the patient over time, to determine that the provisional indication of the neurological deficiency should not be negated.
16. The method of any one of embodiments 11-15, wherein identifying the neurological deficiency comprises:
   based on the determined plurality of neurotransmitter levels for the patient, generating a provisional indication of the neurological deficiency;
   applying one or more rule sets based on the memory information for the patient, including by applying the memory information to a positive emotion to a neurotransmitter (PE-NT) matrix, to determine that the provisional indication of the neurological deficiency should not be negated.
17. The method of any one of embodiments 11-16, wherein the first neurotransmitter is selected from the group consisting of: dopamine, serotonin, testosterone, oxytocin, cannabinoids, and opioids.
18. The method of any one of embodiments 11-17, wherein measuring brain activity of the patient comprises using a functional magnetic resonance imaging (fMRI) scan.
19. The method of any one of embodiments 11-18, wherein the fMRI scan is a 7 Tesla fMRI scan.
20. The method of any one of embodiments 11-19, wherein measuring brain activity of the patient comprises using an electroencephalogram (EEG) scan.
21. The method of any one of embodiments 11-20, wherein measuring brain activity of the patient comprises using functional near-infrared spectroscopy (fNIRs).
22. The method of any one of embodiments 11-21, wherein determining, based on the measured brain activity of the patient, the plurality of neurotransmitter levels for the patient comprises:
   determining one or more locations within the brain for which a signal is to be measured in response to the stimuli;
   determining one or more signal intensities, based on the measured brain activity of the patient, associated with each of the one or more locations of the brain; and
   determining the plurality of neurotransmitter levels based at least in part on the determined one or more locations and on the determined one or more signal intensities.
23. The method of claim 22, wherein determining the plurality of neurotransmitter levels comprises comparing a determined signal intensity for a determined location to a threshold signal intensity level for that location.
24. The method of any one of embodiments 11-23, wherein:
   the plurality of stimuli presented to the patient are analyzed based on the one or more emotions associated with the memories associated with the stimuli, as indicated in the received information, and
   determining the one or more locations within the brain is based at least in part on the one or more emotions indicated in the received information.
25. The method of any one of embodiments 11-24, wherein the one or more emotions comprise an emotion from the group comprising: enthusiasm, sexual desire, pride/recognition, nurturant love, contentment, attachment love, amusement, pleasure, and gratitude.
26. A system for identifying and treating a neurological deficiency, comprising one or more processors configured to cause the system to:
   receive information associated with a patient, wherein the received information includes memory information about a plurality of patient memories, wherein, for each of the plurality of patient memories, the memory information indicates: a respective emotional intensity for each of one or more emotions, respective time information, and respective metadata indicating aspects of the memory;
   provide a plurality of stimuli to the patient, wherein each of the plurality of stimuli are respectively associated with one or more of the patient memories;
   while providing the stimulus to the patient, measure brain activity of the patient, wherein measuring brain activity comprises collecting data indicating location of brain activity and associated intensity of brain activity;
   determine, based on the measured brain activity of the patient, a plurality of neurotransmitter levels for the patient;
   identify, based at least in part on the determined plurality of neurotransmitter levels for the patient, a neurological deficiency with respect to at least a first neurotransmitter for the patient.
27. A non-transitory computer-readable storage medium storing instructions for identifying and treating a neurological deficiency, the instructions configured to be executed by one or more processors of a system to cause the system to:
   receive information associated with a patient, wherein the received information includes memory information about a plurality of patient memories, wherein, for each of the plurality of patient memories, the memory information indicates: a respective emotional intensity for each of one or more emotions, respective time information, and respective metadata indicating aspects of the memory;
   provide a plurality of stimuli to the patient, wherein each of the plurality of stimuli are respectively associated with one or more of the patient memories;
   while providing the stimulus to the patient, measure brain activity of the patient, wherein measuring brain activity comprises collecting data indicating location of brain activity and associated intensity of brain activity;
   determine, based on the measured brain activity of the patient, a plurality of neurotransmitter levels for the patient;
   identify, based at least in part on the determined plurality of neurotransmitter levels for the patient, a neurological deficiency with respect to at least a first neurotransmitter for the patient.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

This application discloses several numerical ranges in the text and figures. The numerical ranges disclosed inherently support any range or value within the disclosed numerical ranges, including the endpoints, even though a precise range limitation is not stated verbatim in the specification, because this disclosure can be practiced throughout the disclosed numerical ranges.

The above description is presented to enable a person skilled in the art to make and use the disclosure, and it is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown but is to be accorded the widest scope consistent with the principles and features disclosed herein. Finally, the entire disclosure of the patents and publications referred in this application are hereby incorporated herein by reference.

1. A method for identifying and treating a neurological deficiency, the method performed by a system comprising one or more processors, the method comprising:
receiving information associated with a patient, wherein the received information includes memory information about a plurality of patient memories, wherein, for each of the plurality of patient memories, the memory information indicates: a respective emotional intensity for each of one or more emotions, respective time information, and respective metadata indicating aspects of the memory;
providing a plurality of stimuli to the patient, wherein each of the plurality of stimuli are respectively associated with one or more of the patient memories;
while providing the stimulus to the patient, measuring brain activity of the patient, wherein measuring brain activity comprises collecting data indicating location of brain activity and associated intensity of brain activity;
determining, based on the measured brain activity of the patient, a plurality of neurotransmitter levels for the patient;
identifying, based at least in part on the determined plurality of neurotransmitter levels for the patient, a neurological deficiency with respect to at least a first neurotransmitter for the patient.

2. The method of claim 1, comprising:
identifying, based on the memory information and based on the identified neurological deficiency, a subset of the plurality of patient memories associated with a positive neurological response as indicated by the brain activity of the patient;
identifying, based on the identified subset of the plurality of patient memories and based on the respective metadata indicating aspects for the identified subset, a first aspect that is common across a plurality of the memories; and
generating and providing a personalized treatment plan for the patient comprising an indication of the first aspect.

3. The method of claim 1, wherein identifying the neurological deficiency comprises:
based on the determined plurality of neurotransmitter levels for the patient, generating a provisional indication of the neurological deficiency;
applying one or more rule sets based on pharmacological and/or clinical biochemical information regarding the patient to determine that the provisional indication of the neurological deficiency should not be negated.

4. The method of claim 1, wherein identifying the neurological deficiency comprises:
based on the determined plurality of neurotransmitter levels for the patient, generating a provisional indication of the neurological deficiency;
applying one or more rule sets based on the memory information for the patient, including by comparing emotional intensity information for the patient to emotional intensity threshold information, to determine that the provisional indication of the neurological deficiency should not be negated.

5. The method of claim 1, wherein identifying the neurological deficiency comprises:
based on the determined plurality of neurotransmitter levels for the patient, generating a provisional indication of the neurological deficiency;
applying one or more rule sets based on the memory information for the patient, including by analyzing changes in emotional intensity information for the patient over time, to determine that the provisional indication of the neurological deficiency should not be negated.

6. The method of claim 1, wherein identifying the neurological deficiency comprises:
based on the determined plurality of neurotransmitter levels for the patient, generating a provisional indication of the neurological deficiency;
applying one or more rule sets based on the memory information for the patient, including by applying the memory information to a positive emotion to a neurotransmitter (PE-NT) matrix, to determine that the provisional indication of the neurological deficiency should not be negated.

7. The method of claim 1, wherein the first neurotransmitter is selected from the group consisting of: dopamine, serotonin, testosterone, oxytocin, cannabinoids, and opioids.

8. The method of claim 1, wherein measuring brain activity of the patient comprises using a functional magnetic resonance imaging (fMRI) scan.

9. The method of claim 8, wherein the fMRI scan is a 7 Tesla fMRI scan.

10. The method of claim 1, wherein measuring brain activity of the patient comprises using an electroencephalogram (EEG) scan.

11. The method of claim 1, wherein measuring brain activity of the patient comprises using functional near-infrared spectroscopy (fNIRs).

12. The method of claim 1, wherein determining, based on the measured brain activity of the patient, the plurality of neurotransmitter levels for the patient comprises:
determining one or more locations within the brain for which a signal is to be measured in response to the stimuli;
determining one or more signal intensities, based on the measured brain activity of the patient, associated with each of the one or more locations of the brain; and
determining the plurality of neurotransmitter levels based at least in part on the determined one or more locations and on the determined one or more signal intensities.

13. The method of claim 12, wherein determining the plurality of neurotransmitter levels comprises comparing a determined signal intensity for a determined location to a threshold signal intensity level for that location.

14. The method of claim 1, wherein:
the plurality of stimuli presented to the patient are analyzed based on the one or more emotions associated with the memories associated with the stimuli, as indicated in the received information, and
determining the one or more locations within the brain is based at least in part on the one or more emotions indicated in the received information.

15. The method of claim 1, wherein the one or more emotions comprise an emotion from the group comprising: enthusiasm, sexual desire, pride/recognition, nurturant love, contentment, attachment love, amusement, pleasure, and gratitude.

16. A system for identifying and treating a neurological deficiency, comprising one or more processors configured to cause the system to:
receive information associated with a patient, wherein the received information includes memory information about a plurality of patient memories, wherein, for each of the plurality of patient memories, the memory information indicates: a respective emotional intensity for each of one or more emotions, respective time information, and respective metadata indicating aspects of the memory;
provide a plurality of stimuli to the patient, wherein each of the plurality of stimuli are respectively associated with one or more of the patient memories;
while providing the stimulus to the patient, measure brain activity of the patient, wherein measuring brain activity comprises collecting data indicating location of brain activity and associated intensity of brain activity;
determine, based on the measured brain activity of the patient, a plurality of neurotransmitter levels for the patient;
identify, based at least in part on the determined plurality of neurotransmitter levels for the patient, a neurological deficiency with respect to at least a first neurotransmitter for the patient.

17. A non-transitory computer-readable storage medium storing instructions for identifying and treating a neurological deficiency, the instructions configured to be executed by one or more processors of a system to cause the system to:
receive information associated with a patient, wherein the received information includes memory information about a plurality of patient memories, wherein, for each of the plurality of patient memories, the memory information indicates: a respective emotional intensity for each of one or more emotions, respective time information, and respective metadata indicating aspects of the memory;
provide a plurality of stimuli to the patient, wherein each of the plurality of stimuli are respectively associated with one or more of the patient memories;
while providing the stimulus to the patient, measure brain activity of the patient, wherein measuring brain activity comprises collecting data indicating location of brain activity and associated intensity of brain activity;
determine, based on the measured brain activity of the patient, a plurality of neurotransmitter levels for the patient;
identify, based at least in part on the determined plurality of neurotransmitter levels for the patient, a neurological deficiency with respect to at least a first neurotransmitter for the patient.
